(19) United States
(12) Patent Application Publication
(10) Pub. No.: US 2025/0302725 A1
(43) Pub. Date: Oct. 2, 2025

(54) COMPOSITIONS AND METHODS FOR IMPROVING THE PHYSICAL PROPERTIES OF HEALTHY AND DAMAGED HAIR

(71) Applicant: KEVIN MURPHY GROUP PTY. LTD., Brisbane, Queensland (AU)

(72) Inventors: Douglas Dean Schoon, Dana Point, CA (US); James Nordstrom, Las Vegas, NV (US); Easley Wallace, JR., Berwyn, PA (US); Howard Slaff, Wilmington, DE (US)

(73) Assignee: KEVIN MURPHY GROUP PTY. LTD., Brisbane, Queensland (AU)

(21) Appl. No.: 18/575,107

(22) PCT Filed: Jul. 6, 2022
(86) PCT No.: PCT/IB2022/000392

§ 371 Date: Dec. 28, 2023

Related U.S. Application Data
(60) Provisional application No. 63/219,560, filed on Jul. 8, 2021.

(51) Int. Cl. A61K 8/81; A61Q 5/12

(52) U.S. Cl. CPC A61K 8/8152; A61Q 5/12

(57) ABSTRACT

A method is described for applying polymerizable compositions containing selected (meth)acrylate monomers to human hair to significantly improve the hair's physical properties, where the method involves temporarily delaying polymerization of the composition for a time sufficient to allow absorption of the composition inside the hair shaft, thereby restricting polymerization of the monomer exclusively to inside the hair shaft, while preventing unwanted polymerization on the outer surface of the hair. The result is hair that is measurably stronger and exhibits an improved physical condition, better manageability, and healthier appearance with greater resistance to future hair damage.

### CROSS-REFERENCE TO RELATED APPLICATIONS

This application claims priority to U.S. Provisional Application No. 63/219,560, entitled COMPOSITIONS AND METHODS FOR IMPROVING THE PHYSICAL PROPERTIES OF HEALTHY AND DAMAGED HAIR, filed on 8 Jul. 2021, the contents of which are incorporated herein by reference in their entirety for all purposes.

### FIELD OF INVENTION

The present application describes thermal-or photo-polymerizable compositions for repairing and/or improving human hair and methods of polymerizing the compositions inside the hair shaft to improve physical properties by repairing internal hair damage most often caused by, but not limited to, environmental exposure, repeated washing, physical abuse such as combing or brushing, and chemical treatment.

### BACKGROUND OF THE INVENTION

Normal hair consists of long chains of protein polymers that are cross-linked to create a strong, internal structure where the polymeric chains are bonded by chemical linkages that include covalent, ionic and hydrogen bonds, and van der Waals forces. Cross-linking via covalent disulfide bonds also contributes to the strength and toughness of the overall hair structure. Despite this durability, hair is prone to degradation over time, resulting in low levels of damage that can accumulate until catastrophic failure occurs. Repeated sunlight exposure, brushing/combing, blow drying, and flat ironing contributes to a large portion of this damage, which manifests itself as splitting, tangling, roughness, and overall dull appearing hair that easily breaks. Many conventional hair grooming products and retail/salon treatments can also create significant amounts of similar damage to hair. Even sunlight exposure results in damage to hair.

A wide range of topical products that superficially hide the most obvious effects of either cumulative or sudden hair damage are on the market in the form of gels, lotions, creams, masques, and other compositions designed to address and to seemingly repair some aspects of the damage, but only in a limited and temporary manner, since typically these effects are lost when the hair is washed. Traditional hair products are focused on creating hair that is healthy in appearance, but the hair is likely to remain in a weakened state and certainly more vulnerable to additional damage. Many existing products focus on increasing the moisture and/or oil content of hair to plasticize and alter its refractive index and thus improve its visual appearance. These treatments are primarily superficial and are designed to temporarily improve the hair's appearance and condition, mainly focusing on improving the outer surfaces of the hair.

With continued poor treatment, the hair's physical condition will continue to degrade, leading to fly-away hair that is difficult to control or manage and that easily splits or breaks, despite regular use of traditional treatments such as hair conditioners or masques.

Chemical and/or physical damage causes structural changes inside the hair shaft, creating internal pits, pockets, spaces, holes, cracks, splits, fractures, and/or voids. Chemical oxidation of the disulfide bonds in the hair protein, due to lightening or bleaching of the hair can cause moderate to catastrophic damage, usually starting with small breaks/cracks forming between cuticle layers and/or between the cuticle layers and the cell membrane complex (CMC). These initial injuries to the hair may ultimately lead to catastrophic failure. Chemical degradation of amino acids such as methionine, tyrosine and threonine is also often a precursor to physical degradation of the hair. Normally, approximately 15-20% of the disulfide cross links in human hair are destroyed during a normal bleaching service, and cumulative treatments or over-bleaching can destroy nearly half of the cross links and possibly more (Robbins, Clarence R., Chemical and Physical Behavior of Human Hair, 5^{th} Ed., Springer, 266-268 (2012)).

In other treatments, the hair fiber is oxidized with hydrogen peroxide and reduced with a thioglycolate salt, which also creates significant and unavoidable internal damage to the hair structure, such as cracks and voids scattered through the cortex. Brushing or otherwise extending wet or dry hair is also a cause of cracking between the endocuticle or cortical cell and the CMC, as well as transverse cracking seen in the outer cuticle layer. Blow driers and heat drying and straightening/smoothing via flat iron also create and exacerbate thermally induced physical damage.

A need exists for a composition that can more effectively mend or repair physically damaged hair and a treatment that can strengthen hair to minimize the harmful effects of potentially damaging insults/abuse that may occur. Physically damaged areas inside the hair shaft would benefit from a treatment that reinforces such areas to make them strong and durable again. Even hair that is in a healthy condition would experience significant improvement in measured physical properties as result of treatment by the compositions of the present invention, especially when applied according to the methods described herein. The compositions provide hair with inherent protection against future assaults that could result in additional internal damage, by repeatedly resisting washing out and increasing the hair's resistance to future damage with cumulative treatments, as described herein. The polymeric and/or oligomeric material formed inside the hair can benefit and improve all types of hair and in any condition ranging from virgin to highly damaged.

Previous attempts to polymerize inside the hair structure were reported as early as 1969, using aqueous monomer solutions applied to hair that was pretreated using tetrakis (hydroxymethyl)-phosphonium chloride (THPC) which disrupted important keratin cross-linking through a nucleophilic attack on the disulfide bonds. In addition, polymerization has been carried out using an aqueous persulfate redox initiation system as described in Wolfram, L. J., et al., J. Soc. Cosmet. Chem. 20, 539-553 (August 1969).

Radical initiation reactions fall into two categories determined by how the initial free radical species is formed: Method (1): homolytic decomposition of covalent bonds, or Method (2): electron transfer from ions or atoms containing unpaired electrons followed by bond dissociation in the acceptor molecule. Method (2), utilized by Wolfram, is an aqueous redox initiation system blended with an acrylamide monomer to achieve polymerization at a pH range of 1.5 to 3.5. All reactions occur below pH 7, because the reaction is inhibited by alkaline pH conditions (Robbins, Clarence, et al., J. Soc. Cosmet. Chem., 25, 525 (1974)). In contrast, the present invention is non-aqueous and relies on (meth)acrylate monomers and organic peroxides which form free-radical initiators via homolytic decomposition of covalent bonds (Method 1).

Previous attempts to polymerize monomers in situ have met with limited success. Wolfram describes his redox initiated reaction as desirable because it is faster and more efficient than conventional techniques. It was determined that methods for polymerization inside the hair can be greatly improved and made more practical by utilizing less efficient and slower thermally driven polymerization initiators such as those that undergo homolytic decomposition. It was discovered that these initiators provide surprisingly longer latency or induction periods before onset of mass polymerization. Alternatively, the use of UV or visible or polychromatic light, photosensitive initiators, and photopolymerizable methacrylates and/or acrylates create oligomers and/or polymers nearly instantly directly inside the hair shaft.

The principles and mechanisms by which UV or visible or polychromatic light containing both UV and visible light can initiate cationic and anionic photopolymerizations with suitable photoinitiating systems are described by Fourassier, et al., Progress in Organic Coatings 47 (2003) 16-36, Elsevier Science B.V. These photopolymerizations require low amounts of energy and may be conducted at room temperature or at elevated temperatures, if desired.

UV and visible light photopolymerizations follow one of two pathways classified as either Norrish Type I or Norrish Type II reactions both of which are useful for this invention.

Polychromatic light photopolymerizations simultaneously activate both Norrish Type I and Norrish Type II photopolymerization reactions.

Norrish Type I reactions proceed by photochemical cleavage or homolysis of aldehydes and ketones into two free radical intermediates. A carbonyl group accepts a photon and is excited to a photochemical singlet state which leads to cleavage of the α-carbon-carbon bond creating two radicals.

Norrish Type II reactions proceed by photochemical intramolecular abstraction of a γ-hydrogen by the excited carbonyl compound to produce a 1,4-biradical as a primary photo product.

Norrish Type II reactions utilize organic amine synergists to serve as a hydrogen donor for this polymerization mechanism.

Subsequent attempts at hair treatments by C. Robbins, et al. also focused on aqueous emulsion polymerization using methyl methacrylate polymerized by cumene hydroperoxide. Robbins describes this diffusion-controlled process as being a dependent chemical reduction to radically decrease the amount of cross-linking of the hair fiber. This reduction was accomplished by destroying large numbers of cross-linking disulfide bonds using 6% ammonium thioglycolate at a pH of 9.2 for 10 minutes or sodium bisulfate. After such treatment, the hair must then be re-oxidized to reform the broken disulfide cross-linking which often leads to additional internal hair damage. Use of reducing agents is likely to induce additional and significant levels of hair damage, which is counterproductive as a hair damage repair/strengthening treatment (Robbins, Clarence, et al., J. Soc. Cosmet. Chem, 25, 497-421 (1974)). Scanning electron micrographs of the surface of the treated hair show a thick coating of polymer on the outside of the hair, which is deemed unacceptable since this would significantly alter the tactile properties of hair, making it feel coated with a plastic synthetic texture (Robbins, Clarence R., Chemical and Physical Behavior of Human Hair, 5th Ed., Springer, 529, (2012)).

U.S. Pat. No. 7,820,147 to Mata describes a method for using one or more hair shaft bonding agents, preferably low molecular weight methacrylic acids or acrylic acids in combination with a solvent, preferably water, and a wetting agent and a thickener with the intent of creating a hair shaft bonding agent. The method involves the use of at least one of several oligomers and polymers, including (meth)acrylic acid oligomers, polymers, and copolymers. However, no polymerization initiators are utilized and therefore no internal hair shaft polymerization is expected, and the Mata composition serves only to function as a bonding agent or glue to bond together separated layers of cuticle and discontinuities near the outer surface, which are rather easy to fill with a sticky, high molecular weight oligomer or polymer. This hair shaft bonding agent action is superficial and is only capable of repairing the outermost levels where hair can be damaged by adhering back together any loosened cuticle layers, rejoining split ends, and filling in gaps where the cuticle layer has been removed completely. Any chemical reactions would be restricted to interactions between the methacrylic or acrylic acid with naturally occurring side chain moieties found in the outer cuticle layers, but with no polymerization of monomers occurring inside the hair shaft.

U.S. Pat. No. 3,676,550 to Anzunio describes a water-based treatment (10-90%) as well, utilizing a critical component in the form of a water-soluble halide salt to create a water solubilized cation which in turn liberates free-radicals to polymerize ethylenically unsaturated vinyl monomers. With this system, the use of heat is mandatory, with a temperature range of 100F-140F being preferable, indicating that these reactions will not proceed until the temperature of the hair is warmed to at least 90F. The hair is first chemically reduced which increases the graft copolymerization reaction with the keratinous substrate, which is an undesirable feature of Anzunio that the present invention seeks to avoid. Reducing disulfide bonds weakens the hair, even when these bonds are later reformed by oxidation.

U.S. Pat. No. 5,362,486 to Nandagiri describes in-situ polymerization of pre-reacted oligomers to create a film or coating on the outside surface of the hair to beneficially alter one or more surface properties of the hair. The Nandagiri composition does not absorb into and polymerize inside the hair, but instead remains on the outside of the hair shaft, where polymers used for cosmetic hair purposes typically are deposited.

### SUMMARY OF THE INVENTION

An aspect of the invention is a hair treatment composition comprising:
at least one (meth)acrylate monomer;
optionally at least one di(meth)acrylate monomer;
optionally at least one trifunctional monomer (such as a tri(meth)acrylate monomer);
optionally a polymerization catalyst and/or synergist;
at least one polymerization initiator;
optionally a solvent diluent/carrier;
optionally a surfactant;
optionally a pH adjuster;
optionally a penetration enhancer;
optionally a carrier;
optionally a colorant or optical brightener; and
optionally a hair conditioning agent,
wherein the total of the (meth)acrylate monomer, the di(meth)acrylate monomer and the trifunctional monomer are present in the composition in an amount of least 20% by weight, and wherein hair treated by the composition shows an improvement of at least 5% in break force and/or energy-to-break and/or elastic modulus and wherein hair treated by the composition shows an improvement of at least 5% in energy to break.

Another aspect of the invention is a method of treating hair, comprising applying the hair treatment composition to hair.

Another aspect of the invention is a kit comprising the hair treatment composition and instructions for use.

In an exemplary embodiment, the composition comprises one (meth)acrylate monomer and one di(meth)acrylate monomer.

In an exemplary embodiment, the composition comprises two (meth)acrylate monomers and one di(meth)acrylate monomer and/or one tri(meth)acrylate monomer.

In an exemplary embodiment, the polymerization catalyst or synergist is present.

In an exemplary embodiment, the polymerization catalyst is an aromatic or aliphatic tertiary amine.

In an exemplary embodiment, the polymerization synergist is an aromatic or aliphatic primary or secondary amine.

In an exemplary embodiment, the UV polymerization initiator is ethyl (2,4,6-trimethybenzoyl)phenylphosphinate, aka TPO-L.

In an exemplary embodiment, the polychromatic polymerization initiator is camphorquinone.

In an exemplary embodiment, the visible light polymerization initiator is camphorquinone.

In an exemplary embodiment, the surfactant is present and is an alkoxylated amide and/or an ethoxylated fatty acid.

In an exemplary embodiment, the penetration enhancer is present.

In an exemplary embodiment, the carrier for the benzoyl peroxide initiator is present and is in a liquid form or a solid form.

In an exemplary embodiment, hair treated by the composition also shows an improvement of at least 5% in elastic modulus.

In an exemplary embodiment, hair treated by the composition also shows an improvement of at least 10% in elastic modulus.

An aspect of the invention is a hair treatment composition comprising:
10 to 99% by weight of at least one (meth)acrylate monomer;
0 to 20% by weight of at least one di(meth)acrylate monomer;
0 to 5% by weight of at least one tri(meth)acrylate monomer;
between 0.1 and up to 5% by weight of a polymerization initiator;
0 to 2% by weight of a polymerization catalyst and/or synergist;
0 to 15% by weight of a surfactant;
0 to 20% by weight of a penetration enhancer;
0 to 5% by weight of an alkalizing agent and/or pH adjuster;
0 to 80% by weight of an organic solvent;
0 to 5% by weight of a colorant and/or optical brightener; and
0 to 50% carrier;
0 to 5% by weight of a conditioning agent,
wherein the total of the (meth)acrylate monomer and the optional di(meth)acrylate and tri(meth)acrylate monomers are present in the composition in an amount of at least 20% by weight, and
wherein hair treated by the composition shows an improvement of at least 5% in energy to break.

In an exemplary embodiment, the hair treatment composition comprises at least one (meth)acrylate monomer; at least one di(meth)acrylate monomer; optionally a polymerization catalyst and/or synergist; a polymerization initiator; optionally at least one trifunctional monomer; optionally a surfactant; optionally a pH adjuster; optionally a penetration enhancer; optionally a carrier; optionally a colorant; and optionally a conditioning agent.

In an exemplary embodiment, the hair treatment composition comprises at least one (meth)acrylate monomer; at least one di(meth)acrylate monomer; at least one trifunctional monomer; optionally a polymerization catalyst and/or synergist; a polymerization initiator; optionally a surfactant; optionally a pH adjuster; optionally a penetration enhancer; optionally a carrier; optionally a colorant; and optionally a conditioning agent.

In an exemplary embodiment, the hair treatment composition comprises at least one (meth)acrylate monomer; optionally at least one di(meth)acrylate monomer; at least one trifunctional monomer; optionally a polymerization catalyst and/or synergist; a polymerization initiator; optionally a surfactant; optionally a pH adjuster; optionally a penetration enhancer; optionally a carrier; optionally a colorant; and optionally a conditioning agent.

In an exemplary embodiment, the hair treatment composition comprises at least one (meth)acrylate monomer; at least one di(meth)acrylate monomer; optionally at least one trifunctional monomer; optionally a polymerization catalyst and/or synergist; a polymerization initiator; a surfactant; optionally a pH adjuster; optionally a penetration enhancer; optionally a carrier; optionally a colorant; and optionally a conditioning agent.

In an exemplary embodiment, the hair treatment composition comprises at least one (meth)acrylate monomer; at least one di(meth)acrylate monomer; at least one trifunctional monomer; optionally a polymerization catalyst and/or synergist; a polymerization initiator; a surfactant; optionally a pH adjuster; optionally a penetration enhancer; optionally a carrier; optionally a colorant; and optionally a conditioning agent.

In an exemplary embodiment, the hair treatment composition comprises at least one (meth)acrylate monomer; optionally at least one di(meth)acrylate monomer; at least one trifunctional monomer; optionally a polymerization catalyst and/or synergist; a polymerization initiator; a surfactant; optionally a penetration enhancer; optionally a carrier; optionally a colorant; and optionally a conditioning agent.

In an exemplary embodiment, the hair treatment composition comprises at least one (meth)acrylate monomer; a polymerization initiator; and least one of a polymerization catalyst and/or synergist; a surfactant; a pH adjuster; a penetration enhancer; a carrier; a colorant; and a conditioning agent.

In an exemplary embodiment, the hair treatment composition comprises at least one (meth)acrylate monomer; a polymerization initiator; and least two of a polymerization catalyst and/or synergist; a surfactant; a pH adjuster; a penetration enhancer; a carrier; a colorant; and a conditioning agent.

In an exemplary embodiment, the hair treatment composition comprises at least one (meth)acrylate monomer; a polymerization initiator; and least three of a polymerization catalyst and/or synergist; a surfactant; a pH adjuster; a penetration enhancer; a carrier; a colorant; and a conditioning agent.

In an exemplary embodiment, the hair treatment composition comprises at least one (meth)acrylate monomer; a polymerization initiator; and least four of a polymerization catalyst and/or synergist; a surfactant; a pH adjuster; a penetration enhancer; a carrier; a colorant; and a conditioning agent.

In an exemplary embodiment, the hair treatment composition comprises at least one (meth)acrylate monomer; a polymerization initiator; and least five of a polymerization catalyst and/or synergist; a surfactant; a pH adjuster; a penetration enhancer; a carrier; a colorant; and a conditioning agent.

In an exemplary embodiment, the hair treatment composition comprises at least one (meth)acrylate monomer; at least one di(meth)acrylate monomer; a polymerization initiator; and least one of a polymerization catalyst and/or synergist; a surfactant; a pH adjuster; a penetration enhancer; a carrier; a colorant; and a conditioning agent.

In an exemplary embodiment, the hair treatment composition comprises at least one (meth)acrylate monomer; at least one di(meth)acrylate monomer; a polymerization initiator; and least two of a polymerization catalyst and/or synergist; a surfactant; a pH adjuster; a penetration enhancer; a carrier; a colorant; and a conditioning agent.

In an exemplary embodiment, the hair treatment composition comprises at least one (meth)acrylate monomer; at least one di(meth)acrylate monomer; a polymerization initiator; and least three of a polymerization catalyst and/or synergist; a surfactant; a pH adjuster; a penetration enhancer; a carrier; a colorant; and a conditioning agent.

In an exemplary embodiment, the hair treatment composition comprises at least one (meth)acrylate monomer; at least one di(meth)acrylate monomer; a polymerization initiator; and least four of a polymerization catalyst and/or synergist; a surfactant; a pH adjuster; a penetration enhancer; a carrier; a colorant; and a conditioning agent.

In an exemplary embodiment, the hair treatment composition comprises at least one (meth)acrylate monomer; at least one di(meth)acrylate monomer; a polymerization initiator; and least five of a polymerization catalyst and/or synergist; a surfactant; a pH adjuster; a penetration enhancer; a carrier; a colorant; and a conditioning agent.

In an exemplary embodiment, the hair treatment composition comprises at least one (meth)acrylate monomer; at least one di(meth)acrylate monomer; at least one trifunctional monomer; a polymerization initiator; and least one of a polymerization catalyst and/or synergist; a surfactant; a pH adjuster; a penetration enhancer; a carrier; and a colorant; and a conditioning agent.

In an exemplary embodiment, the hair treatment composition comprises at least one (meth)acrylate monomer; at least one di(meth)acrylate monomer; at least one trifunctional monomer; a polymerization initiator; and least two of a polymerization catalyst and/or synergist; a surfactant; a pH adjuster; a penetration enhancer; a carrier; a colorant; and a conditioning agent.

In an exemplary embodiment, the hair treatment composition comprises at least one (meth)acrylate monomer; at least one di(meth)acrylate monomer; at least one trifunctional monomer; a polymerization initiator; and least three of a polymerization catalyst and/or synergist; a surfactant; a pH adjuster; a penetration enhancer; a carrier; a colorant and a conditioning agent.

In an exemplary embodiment, the hair treatment composition comprises at least one (meth)acrylate monomer; at least one di(meth)acrylate monomer; at least one trifunctional monomer; a polymerization initiator; and least four of a polymerization catalyst and/or synergist; a surfactant; a pH adjuster; a penetration enhancer; a carrier; a colorant; and a conditioning agent.

In an exemplary embodiment, the hair treatment composition comprises at least one (meth)acrylate monomer; at least one di(meth)acrylate monomer; at least one trifunctional monomer; a polymerization initiator; and least five of a polymerization catalyst and/or synergist; a surfactant; a pH adjuster; a penetration enhancer; a carrier; a colorant; and a conditioning agent.

In an exemplary embodiment, the hair treatment composition comprises at least one (meth)acrylate monomer; at least one trifunctional monomer; a polymerization initiator; and least one of a polymerization catalyst and/or synergist; a surfactant; a pH adjuster; a penetration enhancer; a carrier; a colorant; and a conditioning agent.

In an exemplary embodiment, the hair treatment composition comprises at least one (meth)acrylate monomer; at least one trifunctional monomer; a polymerization initiator; and least two of a polymerization catalyst and/or synergist; a surfactant; a pH adjuster; a penetration enhancer; a carrier; a colorant; and a conditioning agent.

In an exemplary embodiment, the hair treatment composition comprises at least one (meth)acrylate monomer; at least one trifunctional monomer; a polymerization initiator; and least three of a polymerization catalyst and/or synergist; a surfactant; a pH adjuster; a penetration enhancer; a carrier; a colorant; and a conditioning agent.

In an exemplary embodiment, the hair treatment composition comprises at least one (meth)acrylate monomer; at least one trifunctional monomer; a polymerization initiator; and least four of a polymerization catalyst and/or synergist; a surfactant; a pH adjuster; a penetration enhancer; a carrier; a colorant; and a conditioning agent.

In an exemplary embodiment, the hair treatment composition comprises at least one (meth)acrylate monomer; at least one trifunctional monomer; a polymerization initiator; and least five of a polymerization catalyst and/or synergist; a surfactant; a pH adjuster; a penetration enhancer; a carrier; a colorant; and a conditioning agent.

In an exemplary embodiment, the hair treatment composition contains a UV sensitive photoinitiator.

In an exemplary embodiment, the hair treatment composition contains a visible light sensitive photoinitiator.

In an exemplary embodiment, the hair treatment composition contains a polychromatic light sensitive photoinitiator.

In an exemplary embodiment, the hair treatment composition contains two or more of a UV sensitive photoinitiator, a visible light photoinitiator and a polychromatic light sensitive photoinitiator.

In an exemplary embodiment, the hair treatment composition contains a UV sensitive photoinitiator and a heat sensitive organic peroxyl initiator.

In an exemplary embodiment, the hair treatment composition is substantially water free.

In an aspect of the invention, the hair treatment composition is applied as a standalone service to improve hair health and is not in combination with other salon hair services, such as bleaching, tinting, coloring, permanent waving or straightening or curling.

Another aspect of the invention is a method of treating hair, comprising applying the hair treatment composition and a different composition to hair.

Another aspect of the invention is a kit comprising the hair treatment composition and instructions for use.

In an exemplary embodiment, the hair treatment composition comprises one (meth)acrylate monomer and one di(meth)acrylate monomer.

In an exemplary embodiment, the hair treatment composition comprises two (meth)acrylate monomers and one di(meth)acrylate monomer and/or one tri(meth)acrylate monomer.

In an exemplary embodiment, the polymerization catalyst is present.

In an exemplary embodiment, the polymerization catalyst is an aromatic or aliphatic tertiary amine.

In an exemplary embodiment, the polymerization initiator is benzoyl peroxide.

In an exemplary embodiment, the surfactant is present and is an alkoxylated amide and/or an ethoxylated fatty acid.

In an exemplary embodiment, the penetration enhancer is present.

In an exemplary embodiment, the carrier for the benzoyl peroxide initiator is present and is in a liquid form or a solid form.

In an exemplary embodiment, hair treated by the hair treatment composition also shows an improvement of at least 5% in elastic modulus.

In an exemplary embodiment, hair treated by the hair treatment composition also shows an improvement of at least 10% in elastic modulus.

In an exemplary embodiment, the hair treatment composition comprises at least one (meth)acrylate monomer; at least one di(meth)acrylate monomer; a polymerization catalyst and/or synergist; one or more of a UV or visible light and polychromatic photopolymerization initiator; optionally at least one trifunctional monomer; optionally a surfactant; optionally an organic solvent, optionally a pH adjuster; optionally a penetration enhancer, optionally a colorant; and optionally a conditioning agent.

In an exemplary embodiment, the hair treatment composition consists of at least one (meth)acrylate monomer; at least one di(meth)acrylate monomer; a polymerization catalyst and/or synergist; one or more of a UV or visible light and polychromatic photopolymerization initiator; optionally at least one trifunctional monomer; optionally a surfactant; optionally a pH adjuster; optionally a penetration enhancer; optionally a colorant; and optionally a conditioning agent.

In an exemplary embodiment, the hair treatment composition consists of at least one (meth)acrylate monomer; at least one di(meth)acrylate monomer; a polymerization catalyst and/or synergist; two or more of a UV, visible light and polychromatic light photopolymerization initiator, optionally at least one trifunctional monomer; optionally a surfactant; optionally an organic solvent, optionally a pH adjuster; optionally a penetration enhancer; optionally a colorant; and optionally a conditioning agent.

In an exemplary embodiment, the hair treatment composition comprises at least one (meth)acrylate monomer; at least one di(meth)acrylate monomer; optionally at least one trifunctional monomer; optionally a polymerization catalyst and/or synergist; a polymerization initiator; a surfactant; optionally a pH adjuster; optionally a penetration enhancer; optionally a carrier; optionally a colorant; and optionally a conditioning agent.

In an exemplary embodiment, the hair treatment composition comprises at least one (meth)acrylate monomer; at least one di(meth)acrylate monomer; at least one trifunctional monomer; optionally a polymerization catalyst and/or synergist; a polymerization initiator; a surfactant; optionally a pH adjuster; optionally a penetration enhancer; optionally a carrier; optionally a colorant; and optionally a conditioning agent.

In an exemplary embodiment, the hair treatment composition comprises at least one (meth)acrylate monomer; optionally at least one di(meth)acrylate monomer; at least one trifunctional monomer; optionally a polymerization catalyst and/or synergist; a polymerization initiator; a surfactant; optionally a penetration enhancer; optionally a carrier; optionally a colorant; and optionally a conditioning agent.

In an exemplary embodiment, the hair treatment composition comprises at least one (meth)acrylate monomer; a polymerization initiator; and least one of a polymerization catalyst and/or synergist; a surfactant; a pH adjuster; a penetration enhancer; a carrier; a colorant; and a conditioning agent.

In an exemplary embodiment, the hair treatment composition comprises at least one (meth)acrylate monomer; a polymerization initiator; and least two of a polymerization catalyst and/or synergist; a surfactant; a pH adjuster; a penetration enhancer; a carrier; a colorant; and a conditioning agent.

In an exemplary embodiment, the hair treatment composition comprises at least one (meth)acrylate monomer; a polymerization initiator; and least three of a polymerization catalyst and/or synergist; a surfactant; a pH adjuster; a penetration enhancer; a carrier; a colorant; and a conditioning agent.

In an exemplary embodiment, the hair treatment composition comprises at least one (meth)acrylate monomer; a polymerization initiator; and least four of a polymerization catalyst and/or synergist; a surfactant; a pH adjuster; a penetration enhancer; a carrier; a colorant; and a conditioning agent.

In an exemplary embodiment, the hair treatment composition comprises at least one (meth)acrylate monomer; a polymerization initiator; and least five of a polymerization catalyst and/or synergist; a surfactant; a pH adjuster; a penetration enhancer; a carrier; a colorant; and a conditioning agent.

In an exemplary embodiment, the hair treatment composition comprises at least one (meth)acrylate monomer; at least one di(meth)acrylate monomer; a polymerization initiator; and least one of a polymerization catalyst and/or synergist; a surfactant; a pH adjuster; a penetration enhancer; a carrier; a colorant; and a conditioning agent.

In an exemplary embodiment, the hair treatment composition comprises at least one (meth)acrylate monomer; at least one di(meth)acrylate monomer; a polymerization initiator; and least two of a polymerization catalyst and/or synergist; a surfactant; a pH adjuster; a penetration enhancer; a carrier; a colorant; and a conditioning agent.

In an exemplary embodiment, the hair treatment composition comprises at least one (meth)acrylate monomer; at least one di(meth)acrylate monomer; a polymerization initiator; and least three of a polymerization catalyst and/or synergist; a surfactant; a pH adjuster; a penetration enhancer; a carrier; a colorant; and a conditioning agent.

In an exemplary embodiment, the hair treatment composition comprises at least one (meth)acrylate monomer; at least one di(meth)acrylate monomer; a polymerization initiator; and least four of a polymerization catalyst and/or synergist; a surfactant; a pH adjuster; a penetration enhancer; a carrier; a colorant; and a conditioning agent.

In an exemplary embodiment, the hair treatment composition comprises at least one (meth)acrylate monomer; at least one di(meth)acrylate monomer; a polymerization initiator; and least five of a polymerization catalyst and/or synergist; a surfactant; a pH adjuster; a penetration enhancer; a carrier; a colorant; and a conditioning agent.

In an exemplary embodiment, the hair treatment composition comprises at least one (meth)acrylate monomer; at least one di(meth)acrylate monomer; at least one trifunctional monomer; a polymerization initiator; and least one of a polymerization catalyst and/or synergist; a surfactant; a pH adjuster; a penetration enhancer; a carrier; and a colorant; and a conditioning agent.

In an exemplary embodiment, the hair treatment composition comprises at least one (meth)acrylate monomer; at least one di(meth)acrylate monomer; at least one trifunctional monomer; a polymerization initiator; and least two of a polymerization catalyst and/or synergist; a surfactant; a pH adjuster; a penetration enhancer; a carrier; a colorant; and a conditioning agent.

In an exemplary embodiment, the hair treatment composition comprises at least one (meth)acrylate monomer; at least one di(meth)acrylate monomer; at least one trifunctional monomer; a polymerization initiator; and least three of a polymerization catalyst and/or synergist; a surfactant; a pH adjuster; a penetration enhancer; a carrier; a colorant and a conditioning agent.

In an exemplary embodiment, the hair treatment composition comprises at least one (meth)acrylate monomer; at least one di(meth)acrylate monomer; at least one trifunctional monomer; a polymerization initiator; and least four of a polymerization catalyst and/or synergist; a surfactant; a pH adjuster; a penetration enhancer; a carrier; a colorant; and a conditioning agent.

In an exemplary embodiment, the hair treatment composition comprises at least one (meth)acrylate monomer; at least one di(meth)acrylate monomer; at least one trifunctional monomer; a polymerization initiator; and least five of a polymerization catalyst and/or synergist; a surfactant; a pH adjuster; a penetration enhancer; a carrier; a colorant; and a conditioning agent.

In an exemplary embodiment, the hair treatment composition comprises at least one (meth)acrylate monomer; at least one trifunctional monomer; a polymerization initiator; and least one of a polymerization catalyst and/or synergist; a surfactant; a pH adjuster; a penetration enhancer; a carrier; a colorant; and a conditioning agent.

In an exemplary embodiment, the hair treatment composition comprises at least one (meth)acrylate monomer; at least one trifunctional monomer; a polymerization initiator; and least two of a polymerization catalyst and/or synergist; a surfactant; a pH adjuster; a penetration enhancer; a carrier; a colorant; and a conditioning agent.

In an exemplary embodiment, the hair treatment composition comprises at least one (meth)acrylate monomer; at least one trifunctional monomer; a polymerization initiator; and least three of a polymerization catalyst and/or synergist; a surfactant; a pH adjuster; a penetration enhancer; a carrier; a colorant; and a conditioning agent.

In an exemplary embodiment, the hair treatment composition comprises at least one (meth)acrylate monomer; at least one trifunctional monomer; a polymerization initiator; and least four of a polymerization catalyst and/or synergist; a surfactant; a pH adjuster; a penetration enhancer; a carrier; a colorant; and a conditioning agent.

In an exemplary embodiment, the hair treatment composition comprises at least one (meth)acrylate monomer; at least one trifunctional monomer; a polymerization initiator; and least five of a polymerization catalyst and/or synergist; a surfactant; a pH adjuster; a penetration enhancer; a carrier; a colorant; and a conditioning agent.

In an exemplary embodiment, the trifunctional monomer is a tri(meth)acrylate monomer.

In an exemplary embodiment, the (meth)acrylate monomer is a methacrylate monomer.

In an exemplary embodiment, the (meth)acrylate monomer is an acrylate monomer.

In an exemplary embodiment, the di(meth)acrylate monomer is a dimethacrylate monomer.

In an exemplary embodiment, the di(meth)acrylate monomer is a diacrylate monomer.

In an exemplary embodiment, the trifunctional monomer is a trimethacrylate monomer.

In an exemplary embodiment, the trifunctional monomer is a triacrylate monomer.

In an exemplary embodiment, the hair treatment composition contains water in a weight range of 0 to 5%, such as 0 to 3%, such as 0 to 2%, such as 0 to 1%, such as 0 to 0.1%, such as between 0 and 1%, such as between 0 and 5%, such as between 0 and 3%.

In an exemplary embodiment, the hair treatment composition is substantially water free.

In an aspect of the invention, the hair treatment composition is applied as a standalone service to improve hair health and is not in combination with other salon hair services, such as bleaching, tinting, coloring, permanent waving or straightening or curling.

Another aspect of the invention is a method of treating hair, comprising applying the hair treatment composition of the present invention and a different hair treatment composition (such as a commercial hair treatment composition) to hair.

In an exemplary embodiment, the different hair composition is applied to hair before the hair treatment composition of the present invention is applied.

In an exemplary embodiment, the different hair composition is applied to hair after the hair treatment composition of the present invention is applied.

In an exemplary embodiment, the different hair composition is applied to hair at the same time as the hair treatment composition of the present invention is applied.

In an exemplary embodiment, thermal polymerization of the uncured monomeric hair treatment composition is delayed until after absorption of the monomeric treatment composition into the hair occurs, such as for at least long enough to mix and apply the treatment to the hair and then allow for at least 10 minutes, more preferably approximately 30 minutes, or alternatively up to one hour, for the treatment composition to penetrate into the interior of the hair before removal of the treatment composition from the surface of the hair. Thus, in an embodiment, there is an induction period after mixing of the treatment composition of at least one hour or more before the onset of mass polymerization inside the hair structure. In other embodiments, there is an induction period after mixing of the treatment composition of two to three hours. In the present invention, thermal polymerization is prevented from occurring too quickly, to allow sufficient time for deeper penetration of the treatment composition into the interior of the hair before the free-radical reaction transforms the monomers present in the treatment composition into the first initially formed dimers and trimers, etc. Otherwise, polymerization of the treatment composition would primarily and undesirably occur outside of the hair, rather than being allowed to extend deeper into the cortex of the hair.

In another exemplary embodiment, one or more of UV, visible and polychromatic light photopolymerization follows after absorption of the uncured monomeric hair treatment composition into the hair occurs, such as for at least long enough to mix and apply the treatment composition to the hair and then allow for at least 5 minutes, more preferably for approximately 30 minutes, or for 35 to 60 minutes or longer for the treatment composition to penetrate into the interior of the hair before removal of the treatment composition from the surface of the hair. Thus, in an embodiment, there is an induction period after mixing of the treatment composition of at least one hour or more before the onset of mass polymerization of the treatment composition inside the hair structure. In other embodiments, there is an induction period after mixing of the treatment composition of two to three hours. In the present invention, polymerization is prevented from occurring too quickly, to allow sufficient time for deeper penetration of the treatment composition into the interior of the hair before the free-radical reaction transforms the monomers present in the treatment composition into the first initially formed dimers and trimers, etc. Otherwise, polymerization of the treatment composition would primarily and undesirably occur outside of the hair, rather than being allowed to extend deeper into the cortex of the hair.

In another exemplary embodiment, one or more of UV, visible and polychromatic light photopolymerization follows after absorption of the uncured monomeric hair treatment composition into the hair occurs, such as for at least long enough to mix and apply the treatment composition to the hair and then allow for at least 5 minutes, more preferably approximately 30 minutes, or 35 to 60 minutes or longer, for the treatment composition to penetrate into the interior of the hair before removal of the treatment composition from the surface of the hair. The onset of polymerization is controlled through the use of one or more of UV, visible or polychromatic light and one or more UV, visible and polychromatic light sensitive photoinitiators present in the treatment composition. Incorporation of one or more of a UV, visible or polychromatic light sensitive photoinitiator in the treatment composition allows additional time for deeper penetration of the treatment composition into the interior of the hair, rather than having free radical polymerization undesirably occurring before such penetration can occur.

In an exemplary embodiment, one or more different hair treatments (such as coloring, bleaching, etc.) are blended with or combined into the hair treatment composition of the present invention and applied to hair as an integral part of a total hair treatment composition.

In an exemplary embodiment of a method of treating hair by applying to the hair the hair treatment composition of the present invention, the composition is applied to the hair in the presence of an alkalizing ingredient to increase the pH of the composition to at least 8.2.

In an exemplary embodiment of the method, the hair treatment composition of the present application is applied approximately ¼ inch from the scalp.

In an exemplary embodiment of the method, after application of the hair treatment composition of the present invention to hair, the hair shows improvement after contact for at least 10 minutes, such as at least 12 minutes, such as at least 15 minutes, such as at least 20 minutes.

In an exemplary embodiment of the method, after the hair treatment composition of the present application is applied to hair, the hair is contacted for at least 10 minutes, such as at least 12 minutes, such as at least 15 minutes, such as at least 30 minutes and subsequently cured with wavelengths in the UV and visible light range. In an exemplary embodiment of the method, the applied composition is absorbed into the hair (shafts) and is subsequently cured with UV energy, such as in the UV-A region.

In an exemplary embodiment of the method, the applied composition is absorbed into the hair entirely.

In an exemplary embodiment of the method, the applied composition is absorbed into the hair (shafts) and is subsequently cured with visible light energy, such as in the 500-401 visible region.

In an exemplary embodiment of the method, after treatment with the hair treatment composition of the present invention, the hair shows an improvement of at least 5% in elastic modulus.

In an exemplary embodiment of the method, at least a second treatment of the hair with the hair treatment composition of the present invention results in cumulative improvement in the hair's energy-to-break.

In an exemplary embodiment of the method, at least a second treatment of the hair with the hair treatment composition of the present invention results in cumulative improvement in the hair's elastic modulus.

In an exemplary embodiment of the method, the hair is damaged, undamaged, or virgin.

In an exemplary embodiment of the method, the hair has previously undergone an alkaline treatment (i.e., subjecting the hair to a pH of greater than 7).

In an exemplary embodiment of the method, the hair is bleached, colored, tinted or waved.

In an exemplary embodiment of the method, the hair is wet.

In an exemplary embodiment of the method, the hair is dry.

Exemplary embodiments of the hair treatment composition include, but are not limited to,
dimethyl amino methyl propanol, tetraethylene glycol dimethacrylate, 2-(acetoacetoxy)ethyl methacrylate, cetyl methacrylate, ethanol, dimethyl isosorbide, PPG-2-hydroxyethyl cocoamide, ethyl 4-(dimethyl amino)benzoate, benzoyl peroxide, and silica;
dimethyl amino methyl propanol, 1,12-dodecanediol dimethacrylate, stearyl methacrylate, ethanol, dimethyl isosorbide, cocoamide MEA, ethyl 4-(dimethyl amino)benzoate, benzoyl peroxide, and silica;
dimethyl amino methyl propanol, 2-(acetoacetoxy)ethyl methacrylate, cetyl methacrylate, ethanol, dimethyl isosorbide, PPG-2 hydroxyethyl cocoamide ethyl 4-(dimethyl amino)benzoate, benzoyl peroxide, and silica;
dimethyl amino methyl propanol, 1,12-dodecanediol dimethacrylate, 2-(acetoacetoxy)ethyl methacrylate, cetyl methacrylate, ethanol, dimethyl isosorbide, PPG-2 hydroxyethyl coco/isostearamide, ethyl 4-(dimethyl amino) benzoate, benzoyl peroxide, and silica;
dimethyl amino methyl propanol, tetraethylene glycol dimethacrylate, 2-(acetoacetoxy)ethyl methacrylate, cetyl methacrylate, ethanol, dimethyl isosorbide, PPG-2-hydroxyethyl cocoamide, ethyl 4-(dimethyl amino)benzoate, benzoyl peroxide, and silica;
tetraethylene glycol dimethacrylate, stearyl methacrylate, ethanol, dimethyl isosorbide, PPG-2-hydroxyethyl cocoamide, ethyl 4-(dimethyl amino)benzoate, benzoyl peroxide, and silica;
dimethyl amino methyl propanol, cetyl methacrylate, ethanol, dimethyl isosorbide, cocoamide MEA, ethyl 4-(dimethyl amino)benzoate, benzoyl peroxide, and silica;
cetyl/stearyl methacrylate, ethanol, dimethyl 9-decenamide, benzoyl peroxide, and silica;
dimethyl amino methyl propanol, tetraethylene glycol dimethacrylate, 2-(acetoacetoxy)ethyl methacrylate, cetyl methacrylate, ethanol, dimethyl isosorbide, cocoamide MEA, PPG-2-hydroxyethyl cocoamide, ethyl 4-(dimethyl amino) benzoate, benzoyl peroxide, and silica;
dimethyl amino methyl propanol, tetraethylene glycol dimethacrylate, 2-(acetoacetoxy)ethyl methacrylate, cetyl methacrylate, ethanol, dimethyl isosorbide, cocoamide MEA, PPG-2-hydroxyethyl cocoamide, lauric acid, ethyl 4-(dimethyl amino)benzoate, benzoyl peroxide, and silica;
dimethyl amino methyl propanol, tetraethylene glycol dimethacrylate, stearyl methacrylate, ethanol, dimethyl isosorbide, cocoamide MEA, ethyl 4-(dimethyl amino)benzoate, benzoyl peroxide, and silica;
dimethyl amino methyl propanol, tetraethylene glycol dimethacrylate, 2-(acetoacetoxy)ethyl methacrylate, cetyl methacrylate, ethanol, dimethyl isosorbide, PPG-2-hydroxyethyl cocoamide, ethyl 4-(dimethyl amino)benzoate, benzoyl peroxide, and silica;
dimethyl amino methyl propanol, trimethylolpropane trimethacrylate, 2-(acetoacetoxy)ethyl methacrylate, cetyl methacrylate, ethanol, dimethyl isosorbide, PPG-2 hydroxyethyl cocoamide, ethyl 4-(dimethyl amino)benzoate, benzoyl peroxide, and silica;
cetyl/stearyl methacrylate, ethanol, dimethyl 9-decenamide, benzoyl peroxide and acrylic powder;
tetraethylene glycol dimethacrylate, 2-(acetoacetoxy)ethyl methacrylate, cetyl methacrylate, ethanol, dimethyl isosorbide, PPG-2 hydroxyethyl cocoamide, ethyl 4-(dimethyl amino)benzoate, benzoyl peroxide and water;
dimethyl amino methyl propanol, tetraethylene glycol dimethacrylate, 2-(acetoacetoxy)ethyl methacrylate, cetyl methacrylate, ethanol, dimethyl isosorbide, PPG-2 hydroxyethyl cocoamide, benzoyl peroxide, and silica;
dimethyl amino methyl propanol, tetraethylene glycol dimethacrylate, 2-(acetoacetoxy)ethyl methacrylate, cetyl methacrylate, ethanol, dimethyl isosorbide, POE (20) sorbitan monolaurate, ethyl 4-(dimethyl amino)benzoate, benzoyl peroxide, and silica;
dimethyl amino methyl propanol, tetraethylene glycol dimethacrylate, 2-(acetoacetoxy)ethyl methacrylate, cetyl methacrylate, ethanol 200 pf., dimethyl isosorbide, POE (20) sorbitan monolaurate, PPG-2 hydroxyethyl cocamide, ethyl 4-(dimethyl amino)benzoate, phytantriol, TPO-L;
dimethyl amino methyl propanol, tetraethylene glycol dimethacrylate, 2-(acetoacetoxy)ethyl methacrylate, cetyl methacrylate, dimethyl isosorbide, POE (20) sorbitan monolaurate, PPG-2 hydroxyethyl cocamide, ethyl 4-(dimethyl amino) benzoate, phytantriol, TPO-L;
dimethyl amino methyl propanol, tetraethylene glycol dimethacrylate, 2-(acetoacetoxy)ethyl methacrylate, cetyl methacrylate, dimethyl isosorbide, POE (20) sorbitan monolaurate, PPG-2 hydroxyethyl cocamide, ethyl 4-(dimethyl amino) benzoate, phytantriol, TPO-L;
dimethyl amino methyl propanol, tetraethylene glycol dimethacrylate, 2-(acetoacetoxy)ethyl methacrylate, cetyl methacrylate, dimethyl isosorbide, POE (20) sorbitan monolaurate, PPG-2 hydroxyethyl cocamide, ethyl 4-(dimethyl amino) benzoate, phytantriol, TPO-L, camphorquinone;
dimethyl amino methyl propanol, tetraethylene glycol dimethacrylate, 2-(acetoacetoxy)ethyl methacrylate, cetyl methacrylate, dimethyl isosorbide, POE (20) sorbitan monolaurate, PPG-2 hydroxyethyl cocamide, ethyl 4-(dimethyl amino) benzoate, phytantriol, TPO-L, camphorquinone;
dimethyl amino methyl propanol, tetraethylene glycol dimethacrylate, 2-(acetoacetoxy)ethyl methacrylate, cetyl methacrylate, dimethyl isosorbide, POE (20) sorbitan monolaurate, PPG-2 hydroxyethyl cocamide, ethyl 4-(dimethyl amino) benzoate, phytantriol, TPO-L, BPO/silica powder;
dimethyl amino methyl propanol, tetraethylene glycol dimethacrylate, 2-(acetoacetoxy)ethyl methacrylate, cetyl methacrylate, ethanol, dimethyl isosorbide, POE (20) sorbitan monolaurate, PPG-2 hydroxyethyl cocamide, ethyl 4-(dimethyl amino)benzoate, phytantriol, and TPO-L;
dimethyl amino methyl propanol, tetraethylene glycol dimethacrylate, 2-(acetoacetoxy)ethyl methacrylate, cetyl methacrylate, dimethyl isosorbide, POE (20) sorbitan monolaurate, PPG-2 hydroxyethyl cocamide, ethyl 4-(dimethyl amino) benzoate, phytantriol, and TPO-L;
dimethyl amino methyl propanol, tetraethylene glycol dimethacrylate, 2-(acetoacetoxy)ethyl methacrylate, cetyl methacrylate, ethanol, dimethyl isosorbide, POE (20) sorbitan monolaurate, PPG-2 hydroxyethyl cocamide, ethyl 4-(dimethyl amino)benzoate, phytantriol, TPO-L, and camphorquinone;
dimethyl amino methyl propanol, tetraethylene glycol dimethacrylate, 2-(acetoacetoxy)ethyl methacrylate, cetyl methacrylate, ethanol, dimethyl isosorbide, POE (20) sorbitan monolaurate, PPG-2 hydroxyethyl cocamide, ethyl 4-(dimethyl amino)benzoate, phytantriol, and camphorquinone;
dimethyl amino methyl propanol, tetraethylene glycol dimethacrylate, 2-(acetoacetoxy)ethyl methacrylate, cetyl methacrylate, dimethyl isosorbide, PPG-2 hydroxyethyl cocamide, ethyl 4-(dimethyl amino)benzoate, phytantriol, and TPO-L; and
dimethyl amino methyl propanol, tetraethylene glycol dimethacrylate, 2-(acetoacetoxy)ethyl methacrylate, cetyl methacrylate, dimethyl isosorbide, PPG-2 hydroxyethyl cocamide, ethyl 4-(dimethyl amino)benzoate, phytantriol, TPO-L, benzoyl peroxide, and silica.

### BRIEF DESCRIPTION OF THE DRAWINGS

The following figures are illustrative of specific embodiments of the invention but are not intended to otherwise limit the breadth of the invention as described herein.

FIG. 1 illustrates that a wide range of induction times can be achieved, as demonstrated by measuring the rate of change in solution turbidity resulting when varying the pH, as well as the concentrations of the preferred catalyst and initiator, benzoyl peroxide and ethyl 4-(dimethylamino)benzoate. FIG. 1 demonstrates the ability to shorten or increase on-set of mass polymerizations at room temperature.

FIGS. 2A and 2B illustrate the effects of increasing concentrations of the surfactant cocamide MEA on European brown hair tresses, demonstrating that increasing the surfactant concentration increases hair toughness by increasing both the slope of the stress/strain curve, as well as the total area under the curve, where toughness is defined as the capacity of the material to absorb energy prior to catastrophic failure. In particular, FIGS. 2A and 2B demonstrate that exemplary formulas 15 to 17, which show an increased concentration of surfactant, improve surface wetting/penetration on a bleached and unbleached standard tress. The cocamide MEA concentration for each formula is 52−2=0%, 52−1=0.2% and 52-3=1%.

FIG. 3 illustrates the effects of using an added polymer cross-linker to alter hair strength. In particular, FIG. 3 demonstrates the effects of using formulas with increasing cross-linker triethylene glycol dimethacrylate (TEGDMA) concentration on damaged hair where Formulation 18=0% TEGMDA; Formulation 19=8% TEGMDA; Formulation 20=16% TEGMDA; and Formulation 21=untreated bleached standard.

FIG. 4 illustrates the stress/strain results of varying exposure times of the hair treatment composition on bleached hair using a low alkalinity formulation. In particular, FIG. 4 demonstrates the effect of soaking time on penetration using exemplary Formulation 21.

FIG. 5 illustrates controlled mass polymerization utilizing water as the benzoyl peroxide (BPO) carrier as determined by turbidity measurements using exemplary Formulation 23. The sampling rate was 6 times per minute and mass polymerization was observed to begin after 75 minutes and 132 minutes.

FIG. 6 illustrates that similar results are achieved when either a dual cure thermal/UV or UV cure are applied before or after performing a hair coloring service using exemplary Formulations 35A and 35B, demonstrating that salon hair color treatments do not prevent or hinder absorption and polymerization of these formula.

### DETAILED DESCRIPTION OF THE INVENTION

### Definitions

As used herein, the term “induction period” refers to a temporary delay in the mass polymerization process that can range from minutes to hours, preferable more than one hour.

As used herein the term “(meth)acrylate” refers to both the methacrylate and the acrylate versions of a compound. For example, methyl (meth)acrylate refers to both methyl methacrylate and methyl acrylate.

As used herein, the term “processing time” refers to the time allotted for compositions to adequately absorb into hair.

As used herein, the term “on-head applications” refers to an application of a product to human hair that remains attached to the scalp or otherwise affixed to a person's head, e.g., hair extensions.

As used herein, the term “off-head hair” refers to hair that is not attached to the head.

As used herein, the term “off-scalp application” refers to an application of a product to human hair that is applied only to the hair not attached to the scalp and therefore avoids product contact with a human scalp.

As used herein, the term “standalone service” refers to an application of a product to human hair individually as a treatment and not in conjunction with other chemical services performed the same day.

As used herein, the term “alkalizing ingredients” or “alkalizer” refers to a substance used to alter or shift the pH of human hair toward an alkaline environment, i.e., greater than pH 7.

As used herein, the term “polymerization initiator” refers to any type of initiator, such a thermal initiator or a photoinitiator that causes polymerization of the monomer-containing (or uncured) hair treatment compositions of the present invention.

As used herein, the term “ultraviolet” or “UV” refers to electromagnetic radiation with wavelengths ranging from 10 to 400 nanometers (nm).

As used herein, the term “visible light” or “Vis” refers electromagnetic radiation with wavelengths ranging from 401 to 700 nanometers (nm).

As used herein, the term “ultraviolet A” or “UV-A” refers to electromagnetic radiation with wavelengths ranging from 315 nm to 400 nanometers (nm).

As used herein, the term “polychromatic light” refers to light containing both UV and visible light.

As used herein, the term “UV photoinitiator” or “UV sensitive photoinitiator” refers to a compound that produce radicals when exposed to wavelengths of electromagnetic radiation in the ultraviolet range.

As used herein, the term “photoinitiator” refers to a compound that produces radicals when exposed to specific wavelengths of electromagnetic radiation in the visible range.

As used herein, the term “full spectrum” refers to the spectrum of light emitted from the sun that reaches the surface of the Earth. Full spectrum light sources are polychromatic devices designed to emulate the full spectrum of light including both visible and UV and possessing a color rendering index of 60-100.

As used herein, the term “UV/Vis” photoinitiator refers to a compound that produces radicals when exposed to specific wavelengths of electromagnetic radiation in the UV and/or visible light range.

As used herein, the term “UV-A photoinitiator” refers to a compound that produces radicals when exposed to wavelengths in the UV-A region of the electromagnetic spectrum.

As used herein, the terms “catalyst” and “synergist” refers to compounds that accelerate photopolymerizations. A catalyst functions by lowering the activation energy to provide a lower energy pathway for polymerization. Synergists are also catalysts but function by lowering the energy gap between the highest occupied molecular orbit (HOMO) and the lowest unoccupied molecular orbit (LUMO). Synergists activate both the nucleophile and the electrophile simultaneously. Catalysts and synergists can be used alone or in combination for possible synergistic effects and/or to create a multi-catalyst reaction mechanism.

As used herein, the phrase “stress-strain curve” refers to a graphical representation of the behavior of a human hair when it is subjected to a tensile load or force.

As used herein, the phrase “stress” refers the ratio of the load or force to the cross-sectional area of a hair strand, typically measured in Newtons per square meter squared.

As used herein, the phrase “strain” refers to a unitless number that represents the ratio of the deformation of the hair sample because of the applied force.

As used herein, the phrase “energy to break” (EB) refers to energy required to break the hair strand and is a measure of the total area under the stress-strain curve for the hair strand and is measured in Joules.

As used herein, the phrase “elastic modulus” (EM) refers to Young's Modulus, the ratio of longitudinal stress to strain which is the ratio of stress to the corresponding strain and a measure the rigidity or stiffness of a material. In terms of the stress-strain curve, the modulus of elasticity is the slope of the stress-strain curve in the range of linear proportionality of stress to strain and is measured in Pascals.

As used herein, the phrase “break force” (BF) refers to the breaking strength and is amount of stress required to reach the fracture/breaking point for a hair strand sample and is measured in Newtons per square meter squared.

As used herein, the term “toughness” (T) refers to Energy-to-Break or the total energy required to stretch and break a hair strand is measured in Pascals.

As used herein, the phrase “cumulative improvement” refers to the continued improvement to the hair that is observed as the result of sequential treatments with the hair treatment composition.

Disclosed are compositions and methods for repairing and improving the internal structures of human hair, which is generally described as fine, usually cylindrical, thread-like strands of keratinous protein growing from follicles in the skin. Human hair, hereinafter also referred to as “hair”, can be found in abundance growing from the scalp and may be removed for use or treatment as loose single strands or bound into the form of a full wig or hair piece or a single tress, a strand, a curl or hair extension or other shapes, styles, or forms.

The hair treatment compositions disclosed herein are reactive and comprise blends of (meth)acrylate monomers formulated to absorb into human hair and then polymerize within the hair shaft, thereby trapping the polymerized composition. The polymerized material inside the hair shaft imparts improved physical properties to the hair such as, but not limited to, improved tensile strength (also known as break force) increased energy to break and elastic modulus, and enhanced textural/sensorial properties of the hair, including improved appearance, better shine/gloss, smoother feel, and easier combing/brushing and shaping with reduced or no static build-up in the hair.

In general, energy to break is improved by at least 5%, such as at least 10%, such as at least 15%, such as at least 20%, such as at least 25%, such as in a range from 5% to 35%, such as from 5% to 30%, such as from 5% to 25%, such as from 5% to 20%, such as from 5% to 15%, such as from 10% to 25%, such as from 10% to 20%, such as from 10% to 15%. Similarly, in general, a single treatment improves elastic modulus by at least 5%, such as at least 10%, such as at least 15%, such as at least 20%, such as at least 25%, such as in a range from 5 to 35%, such as from 5% to 30%, such as from 5% to 25%, such as from 5% to 20%, such as from 5% to 15%, such as from 10% to 25%, such as from 10% to 20%, such as from 10% to 15%.

The hair treatment compositions of the present invention may further contain one or more additional ingredients to facilitate improved absorption into the hair and to ensure subsequent polymerization inside the hair shaft within a period of time considered reasonable by most users. The polymerized oligomer and/or polymer immediately becomes constrained inside the hair shaft, allowing these compositions to provide both immediate and long-lasting benefits after only one application, the benefits of which continue for at least several days, preferably one month, likely significantly longer, possibly becoming a permanent improvement to the physical structure of the hair. Also, the hair treatment composition provides cumulative benefits, improving the hair with each additional treatment as demonstrated in Table 2.

In some embodiments, the method for using the hair treatment compositions is as a standalone treatment applied to any hair type or, alternatively, is included as a part of an existing hair treatment such as, but not limited to, bleaching, permanently waving, curling, straightening, coloring, highlighting, and may be applied to either wet hair or dry hair.

In some embodiments, the method for using the hair treatment compositions is as a treatment applied to a wig, tress, extension or other type of human hair pieces that is off the head and may be used as a standalone treatment or as part of an existing hair treatment such as, but not limited to, bleaching, permanently waving, curling, straightening, coloring, highlighting, and may be applied to either wet hair or dry hair.

The hair treatment compositions are based on reactive (meth)acrylate monomers that absorb into the hair when applied to the hair exterior and then later undergo free radical polymerization inside the hair shaft. The polymerization occurs by chemical conversion of the monomers into short chain oligomers with a molecular weight (MW) range of >500 and <20,000, such as >1,000 and <15,000, such as >1,500 and <10,000, such as >2,000 and <10,000; and long chain polymers with a molecular weight (MW) range of >20,000, such as >40,000, such as >80,000, such as >100,000, such as >1,000,000 with both the short chain oligomers and the long chain polymers being entrapped inside the hair shaft and not capable of escape.

Reactive monomers and the other components determined to be safe for such uses are delivered inside the hair shaft directly by several mechanisms that include:
A). absorption through the intact cuticle layer,
B). absorption between intact cuticle layers,
C). absorption through a damaged cuticle structure,
D). absorption between raised/swollen/separated cuticles,
E). absorption into cracked, split and/or broken hair shafts.

There is no need for pretreatment of the hair with a reducing agent, to artificially increase hair porosity by breaking disulfide cross-links, which undesirably weakens the hair shaft. Further, no keratin reducing agents are required to penetrate and polymerize inside the hair shaft. The hair treatment compositions are suitable for both high and low porosity hair as demonstrated in Table 1.

The hair treatment compositions (also referred to herein as hair treatment formulations) and methods for the long-term improvement of selected physical properties of both healthy and damaged human hair are described herein, including the use of compositions containing selected (meth)acrylate monomer(s), in combination with agents that promote absorption of the monomers into the hair and then, once inside, to trigger free-radical induced mass polymerization of the monomers. The hair treatment compositions unexpectedly impart immediate, significant, and noticeable improvement to the quality of the treated hair. Sensorial improvements include, but are not limited to, increased shine, smoothness, thicker and stronger feel, faster dry times, improved manageability, and lower amounts of static surfaces charges (also known as fly-a-way hair). Other benefits are revealed through the use of established standard scientific methodologies that demonstrate significant improvements in several well-established stress/strain related physical properties of hair, including a) Elastic Modulus (EM), b) Break Force (BF) and c) Energy-to-Break (EB) (also known as Toughness) (see, e.g., Robbins, C.R., Chemical and Physical Behavior of Human Hair, 5th ed., pp. 538-640, Springer, 2012).

The hair treatment compositions described herein may be used as standalone treatments to improve the quality of hair and to save time by incorporation of the compositions as a new additional step in, for example, hair bleaching, permanently waving, curling, straightening, coloring, and highlighting treatments/services.

### Polymerization Reactions

When (meth)acrylate monomers were exposed to certain peroxide-type initiators and suitable catalysts and other conventional initiators at a range of concentrations, it was surprisingly discovered that polymerizations could be carefully controlled and precisely tailored to deliver quick curing inside the hair shaft after a suitably long latency or induction period, thus making the compositions useful for hair treatment services of healthy or damaged hair. The induction period allows sufficient time for the hair treatment compositions to be mixed, applied, and absorbed, a process which can typically require up to one hour when performed, for example, by licensed cosmetologists and on clients in a salon setting.

By way of example, delayed mass polymerization of the hair treatment composition after mixing, i.e., after an “induction period”, can be achieved by adjusting/controlling and thereby delaying on-set of mass polymerization of the monomer components of the composition from one hour up to four hours or longer, such as around two hours or three hours or four hours, preferably around three hours, without adversely impacting the ability of the compositions to polymerize inside the hair shaft. Controlling the induction time provides sufficient time for mixing/blending and applying the compositions to the hair, as well as to allow sufficient time for absorption, i.e., processing time. However, the application and processing times required for commercial off-head application processes may be shortened as required, and the induction time can be desirably reduced to minutes, rather than hours by increasing the concentration of the initiator and/or catalyst. This is readily accomplished through selection of a catalyst with low initial reactivity and controlling its concentration to manipulate the length of the induction period by delaying or accelerating the onset of mass polymerization.

When the selected ingredients are used within selected concentration ranges after the induction period, there is an on-set or rapid spike in the rate of free radical polymerization at room temperature without the need to apply any additional heat. However, increasing the temperature of the composition, once blended, and activated, will also result in additional significant increases in the polymerization rate, almost immediately.

Not limited to these examples, useful heat sources for increasing the polymerization rate include portable or stationary hair drying devices which utilize moving heated air to dry hair or smaller hand-held blow dryers, flat irons, curling irons, as well as infrared (IR) heat lamp units and other similar sources for heat that are useful for drying hair. Artificial heat sources are useful for increasing polymerization rates when the temperatures are within a range from 90F/32C to 450F/232C, such as from 110F/43C to 150F/66C, which falls within the normal working range of most hand-held blow dryers for hair. Room temperatures from 60F/16C to 89F/32C will normally provide sufficient heat to the composition inside the hair shaft, but this extends the onset of the mass polymerization process from minutes to hours. Any drying method which increases the temperature of the hair from room temperature up to the upper limit for hair of about 450F/232C, more preferably no higher than 400F/204C, will also significantly increase the rate of polymerization of the hair treatment composition inside and outside the hair.

When (meth)acrylate monomers are blended with certain ultraviolet or visible or polychromatic light sensitive photoinitiator molecules, an observed advantage is that photopolymerizations can be delayed for long as necessary to allow for increased absorption of the monomeric composition by hair, followed by nearly instantaneous curing inside the hair shaft with relatively brief exposure to UV, visible or polychromatic light of the wavelengths and intensity required for the UV, visible or polychromatic light photoinitiator(s) in the curing composition.

Polymerization can occur at normal room temperatures when the composition is exposed to the wavelengths and intensities required to activate the photoinitiator, with or without the application of additional heat, e.g., to accelerate absorption of the composition.

Any UV/Vis emitting devices which do not cause unsafe skin or eye exposure to the user may be suitably used to polymerize the composition after it has been absorbed into the hair, as long as the device emits the correct UV or visible or polychromatic light wavelengths at useful intensities with sufficient exposure time.

Any wavelength of UV or visible light or combinations thereof can be used as long as it causes the photoinitiator to trigger photopolymerization. Many UV and visible light and polychromatic sources are available with significant emissions near 500, 405, 400, 395 or 380 nm, which matches the absorption spectra of many photoinitiators considered useful for initiating photopolymerization.

A preferred UV emission range is 380 nm+/−5 nm due to the ability of shorter wavelengths to penetration more deeply into darkly colored hair.

A preferred range of visible light is 500-405 nm, which matches commonly used visible light photoinitiators, such as the aliphatic diketones, camphorquinone.

A preferred range of polychromatic light is 375-500 nm, which matches the combined absorption when both UV and visible light photoinitiators are used, such as the combination of Norrish Type I and Norrish Type II photoinitiator, including but not limited to ethyl (2,4,6-trimethylbenzoyl) phenyl phosphinate and camphorquinone.

Multirange wavelength photopolymerizations are also possible, e.g., those with absorption maxima at or near 470 nm/405 nm or 405 nm/380 nm or 380 nm/395 nm or 395 nm/400 nm or 380 nm/400 nm, etc., especially when two or more photo initiators are utilized in the composition.

Absorption into Hair

Consumers have rather low expectations when it comes to hair products that claim to repair hair and most often must settle for conditioning effects that attempt to reverse the appearance of hair damage and other problems related to hair damage, such as brittleness. U.S. Pat. Nos. 9,713,583 and 9,095,518 describe chemical reversal of hair damage by removing cysteic acid residues to allow naturally occurring cross-linkable thiol (—SH) amino acid moieties to reform via air oxidation. These types of products are based on building new cross-linking bonds or repairing/restoring existing cross-linking bonds associated with keratin proteins.

The present invention differs significantly from the above technologies in both form and function by creating a new and unique class of hair treatments that utilizes active free-radical induced, monomer polymerization in situ and entirely within the hair shaft, i.e., a chemical technology previously unavailable in hair care products.

The present invention provides a novel way to treat human hair utilizing reactive monomers selected from compounds deemed to be safe for this application and having specific physicochemical properties that facilitate absorption in hair.

Primary criteria/factors related to increasing monomer absorption include:
1) the selection of monomers having the appropriate chemical properties with acceptable safety profiles and suitable reactivities to facilitate more rapid migration into the hair shaft where prior to polymerizing, the monomers can congregate/pool inside small cracks, spaces, separations, or other types of voids located throughout the hair shaft.
2) the selection of UV or visible light or polychromatic photoinitiator(s) having the appropriate chemical properties with acceptable safety profiles and suitable reactivities to facilitate polymerization of the UV, visible or polychromatic photopolymerizable monomers that congregate/pool inside small cracks, spaces, separations, or other types of voids located throughout the hair shaft.
3) the use of suitable penetration enhancing agents, surfactants, and solvents to facilitate wetting and surface cleansing to improve hair absorption of reactive monomers and other necessary ingredients.
4) the use of alkalizing ingredients to swell open the outer cuticle layers to increase available surface area, thereby allowing increased penetration of the reactive monomers and other necessary ingredients in the described compositions, without reduction of existing disulfide bonds.

Alkalizing ingredients, although not required, are utilized to swell the hair shaft to facilitate increased ease of absorption between the normally compact layers of cuticle cells. Any conventional cosmetic alkalizing ingredients for swelling hair can typically be used as long as the alkalizing agent is soluble in the composition, provides the desired pH range, and does not negatively impact the polymerization process or cause other unwanted effects. Alkalizing agents lacking strong, unpleasant odors and having a good safety profile are preferred.

In an exemplary embodiment, the composition absorption rate is controlled and maximized by using an alkalizing ingredient(s) to increase the pH of the formulation to 8.2 or higher, such as 8.5 or higher, such as 9.0 or higher, such as 9.5 or higher, such as 10.0 or higher, which consequently also increases the pH of the hair shaft. Absorption still occurs at pH values lower than approximately 8.2 due to the particular physicochemical properties of the monomers selected but typically occurs at a slower rate. The ideal pH range of the compositions is determined by examining the type of hair for which it will be the most effective, which ranges from virgin hair to extremely damaged hair. All types of hair can be effectively treated by ensuring that the compositions fall within an appropriate pH range that is suitable for both the hair type and its condition. Damaged hair typically is treated with compositions having lower pH values than virgin hair, even though polymerization inside the hair will occur under both acidic or alkaline conditions. Although not required, the purpose for manipulating the pH is to improve absorption of the monomers, while minimizing the potential for damaging effects to the hair caused by exposure to excessively high pH. Above pH 10, the hair swells excessively which may lead to severe hair damage. A hair shaft will display minimum swelling behavior as the pH is raised from 4 to 9, and more rapid swelling from pH 9 to 10, with a significant increase above pH 10 due to ionization of diacidic amino acid residues and keratin hydrolysis. At PH levels at 10 or higher, hair swelling can reach or exceed 200%, effectively destroying the hair (Robbins, Clarence R., Chemical and Physical Behavior of Human Hair, 5th Ed., Springer, pg. 615-618 (2012)).

Although absorption of the monomers and their subsequent polymerization inside hair can occur at any pH considered safe for the hair, which is approximately pH 3 to 10, a preferred range is pH 7.0 to 10.0, such as pH 7.0 to 9.5, such as pH 7.5 to 10.0, such as pH 7.5 to 9.5, such as pH 8.0 to 10.0, such as pH 8.0 to 9.5, such as pH 8.0 to 9.0, depending on the porosity of the treated hair. When the composition is used following an alkaline cosmetic hair service such as lightening, coloring, straightening, and/or smoothing, the hair is already alkaline, and therefore the open cuticle structure would allow rapid penetration of the monomer components of the composition, thus eliminating the need for an alkalizer to alter the pH in this embodiment.

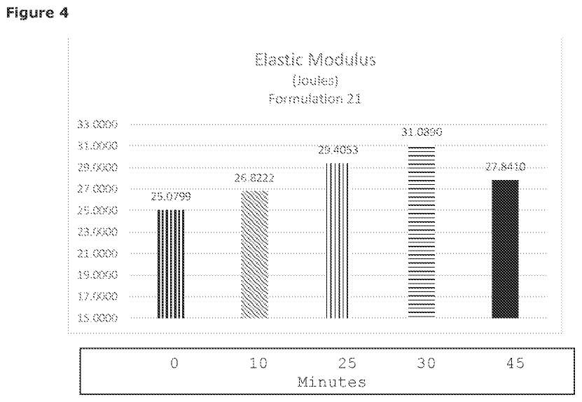

Absorption of the monomers and their subsequent polymerization inside the hair shaft do occur at pH conditions below 7.0. Under these acidic pH conditions, which can range in an exemplary embodiment from pH 3.0 to 6.9, such as pH 3.3 to 6.7, such as pH 3.5 to 6.5, there is typically no alkalizing agent present in the hair treatment composition. It was observed that as the pH of the composition is increased from acidic to alkaline, the hair shaft increasingly swells, allowing the cuticle layers to swell and separate, thus facilitating quicker absorption of the hair treatment composition into the hair shaft to allow maximum saturation to occur over a short period. As a result, alkaline pH values are preferred. Typically, the time required for the hair treatment composition under alkaline conditions (i.e., at a pH greater than 7.0) to absorb into the hair is greater than 5 minutes and less than one hour, such as 5 to 30 minutes, such as 5 to 25 minutes, such as 5 to 20 minutes, such as 5 to 15 minutes, such as 5 to 10 minutes. As demonstrated by FIG. 4, similar trends are observed when higher alkalinity formulations are utilized.

Since visibly damaged hair has a high degree of porosity, absorption occurs more readily when compared to virgin hair with little or no visible damage. In one embodiment, the hair treatment composition is used on relatively high porosity hair and therefore lower alkaline pH formulations can be employed. In another embodiment, the composition has a higher alkaline pH, making it more useful for hair with relatively low porosity, including virgin and some types of ethnic hair.

Known penetration enhancers for keratin can be used as well and are suitable to enhance penetration of the hair treatment composition into hair. Various organic solvents can be used to lower bulk viscosity to enhance keratin penetration. Organic solvents or surface-active agents (surfactants) can also be used to improve absorption of the hair treatment composition by disrupting, dissolving, and thereby removing surface oils/films on the hair exterior to further facilitate enhanced penetration. Should the treatment be used following an alkaline cosmetic hair service such as lightening, coloring, straightening, smoothing, the hair is already alkaline, therefore the open cuticle structure would allow rapid penetration of the active ingredients in this invention, thus eliminating the need for a penetration enhancer.

### Hair Application

Several methods are useful for applying the hair treatment compositions to hair. The compositions may be applied without causing interference with any known cosmetic hair treatments including, but not limited to, bleaching/lightening, coloring, tinting, highlighting, frosting, waving, weaving, curling, relaxing, styling, straightening, smoothing, conditioning, weaving, and tinting. The compositions may be applied immediately before or following such hair treatments. Prior to application, the inventive compositions can also be combined with other known cosmetic hair treatments, thereby simplifying the application.

In one embodiment, the hair treatment compositions are applied as standalone treatments to wet hair, while in another standalone embodiment, the same compositions are applied to dry hair, with no additional changes required to the application procedure, as demonstrated in Tables 3A and 3B.

In another embodiment, the hair treatment composition is applied to the hair immediately before bleaching/lightening treatments or more preferably, immediately following rinsing such treatments from the hair and before finishing by cleansing/shampooing/conditioning the hair.

In another embodiment, the hair treatment composition is applied to the hair immediately before coloring/tinting treatments or more preferably, immediately following rinsing such treatments from the hair and before finishing by cleansing/shampooing/conditioning the hair.

In another embodiment, the hair treatment composition is applied to the hair immediately before chemically relaxing, straightening, or smoothing treatments or more preferably, immediately following rinsing such treatments from the hair and before finishing by cleansing/shampooing/conditioning the hair.

In another embodiment, the hair treatment composition is applied to the hair immediately before other chemical treatments including, permanently waving, curling, smoothing, straightening, or more preferably immediately following rinsing such treatments from the hair and before finishing by cleansing/shampooing/conditioning the hair.

In each embodiment, the hair treatment compositions are applied to the hair as a free-flowing, mobile liquid and then combed or brushed through to ensure a more even distribution. The composition may also be worked into the hair using gloved fingers to prevent excessive skin contact and to reduce the potential for skin irritation while applying the compositions.

There are a wide range of conventional application methods/techniques that may be used to apply the described hair treatment compositions. One non-limiting example is to apply the composition directly onto the hair by squeezing it from a flexible wall container with a suitable applicator tip useful for controlling the flow of the composition. This applicator can be utilized on wet or dry hair as standalone treatments, and before or after chemical treatments such as bleaching or coloring, smoothing, etc.

In an embodiment, a small mixing bowl and tint brush are used to brush the composition directly onto the hair, using the brush bristles to press-in and spread the mobile liquid between the strands for improved coverage. In another embodiment, the composition is applied by spraying or dispensing by pipet/dropper or similar type of application. Hair may also be dipped into a container holding the liquid hair treatment composition or transported to and from the container by a mechanical mechanism.

One preferred method for applying the hair treatment composition to on-head hair is to use a squeeze-type plastic container with a dispensing closure/cap followed by combing the dispensed composition through the hair with a comb or brush. The composition can then be easily applied to a narrow section of hair using a squeeze bottle with an adjustable dispensing tip. The portioning of the flowable composition onto the hair is easily controlled by applying pressure to the flexible container walls, allowing the composition to be applied in a thin stream or line crisscrossing down the length of a narrow, preferably one to two-inch wide hair section, after which the flowable liquid composition is more evenly dispersed by combing or brushing, thus ensuring more complete hair strand wetting and coverage. The application and dispersing process is repeated until the treatment is completed and all or most of the hair strands have been coated with the composition.

One method for applying the hair treatment composition to on-head hair is to use a spray-type plastic container to spray the liquid composition, into the previously sectioned hair, all while combing the dispensed composition through the hair with a comb or brush. The dispersing process is repeated until the treatment is completed and all or most of the hair strands have been coated with the composition.

These hair treatment compositions may be used as on-scalp or off-scalp. It is preferred to use the compositions as off-scalp treatments to minimize the potential for adverse skin reactions, for example in one embodiment by applying the composition approximately one-quarter (14) inch from the scalp. In other embodiments, the composition may be applied at equally suitable distances from the scalp such as one-half (12) inch, one-eighth (18) inch and all distances in between.

A typical, non-limiting example on how the hair treatment composition is applied is as follows:
(a) after washing the hair, rinse with warm water and divide the hair into three sections, two sides, one across the back;
(b) at the basin or chair, apply hair treatment composition with a squeeze bottle onto wet hair, approximately ¼ inch from the scalp;
(c) using a comb or brush and gloved fingers to work through the wet hair to saturation (avoid scalp contact);
(d) cover head with a salon cap or plastic wrap for 30 minutes;
(e) completely rinse away remaining hair treatment composition with warm water until the water runs clean;
(f) wash hair with cleanser/shampoo, rinse with warm water;
(g) condition, rinse and blow dry;
(h) adequately expose treated hair to the (a) elevated temperatures and or (b) correct wavelengths and intensity of UV or visible or polychromatic light, for the time required to cause the absorbed composition to polymerize inside the hair shaft, said exposure from such as with these non-limiting examples; full spectrum lamp, compact fluorescent lamp aka CFL or light emitting diode aka LED UV incorporated into the form of wand, pen, flashlight or into a cap, hat, comb, brush, flatiron, flexible rods or tubes, curlers, waving/curling iron, hair dryer, etc.

Alternatively, the composition can be applied directly to dry hair, skipping step (a) (above) or step (a) may be substituted for another chemical hair treatment or procedure, such as coloring or bleaching/lightening, relaxing, curling, straightening, etc. In such cases, the color or bleaching component is first rinsed with water from the hair and the hair treatment composition is then applied following at least a portion of steps (b) through (h).

Conversely, another non-limiting example would be to perform steps (a) through (h) prior to providing another chemical treatment such as bleaching/lightening, coloring/tinting, relaxing, straighten, smoothing, etc. and then applying the hair treatment composition to either wet or dry hair.

In an exemplary embodiment, steps (a) through (h) occur prior to providing another chemical treatment such as bleaching/lightening, coloring/tinting, relaxing, straighten, smoothing, etc. However, prior to shampooing, a hair treatment composition is applied that contains conventional conditioning agents known to those skilled in the art to improve the final appearance of the hair after subsequent shampooing.

In an exemplary embodiment, steps (a) through (e) occur prior to providing another chemical treatment such as bleaching/lightening, coloring/tinting, relaxing, straighten, smoothing, etc., followed by shampooing and a hair treatment composition is applied that contains conventional conditioning agents known to those skilled in the art to improve the final appearance of the hair after subsequent shampooing.

### UV, Visible and Polychromatic Photopolymerization

Reactive single, double and/or triple functionality (meth)acrylate monomers in amounts of at least 20% by weight (total), such as 20% to 99% by weight, such as 20% to 90% by weight, such as 20% to 85% by weight, such as 20% to 80% by weight, such as 50% to 99% by weight, such as 50% to 95% by weight, such as 50% to 90% by weight, such as 50% to 80% by weight, such as 60% to 99% by weight, such as 60% to 95% by weight, such as 60% to 90% by weight, 70% to 99% by weight, such as 70% to 95% by weight, such as 80% to 99% by weight, are used in combination with UV or visible or polychromatic photopolymerization initiators such as initiator molecule which decomposes to create two organic peroxyl free radicals which initiate self-propagating chain reaction that leads to the creation of an extended oligomer and/or polymer chain. In an exemplary embodiment, the (meth)acrylate monomer may be present in an amount ranging from 10% to 98% by weight, such as 20% to 98% by weight, such as 30% to 98% by weight, such as 30% to 90% by weight, such as 30% to 80% by weight, such as 30% to 70%, such as 40% to 98% by weight, such as 50% to 98% by weight, such as 60% to 98% by weight, such as 70% to 98% by weight. In an exemplary embodiment, the di(meth)acrylate monomer may be present in an amount ranging from 0% to 20% by weight, such as 5% to 20% by weight, such as 10% to 20% by weight, such as 5% to 15% by weight, such as 5% to 10% by weight. In an exemplary embodiment, the tri(meth)acrylate monomer may be present in an amount ranging from 0% to 5% by weight, such as 1% to 5% by weight, such as 3% to 5% by weight, such as 1% to 3% by weight. A single type of monomer may be used to produce homopolymers or alternatively, may be used in combinations of two or three types of monomers to create copolymers or block copolymers or terpolymers or triblock polymers. Additional types of monomers may also be incorporated to create multipolymer and/or multiblock polymers of more complex compositions, e.g., quadpolymers or quadblock polymers, etc.

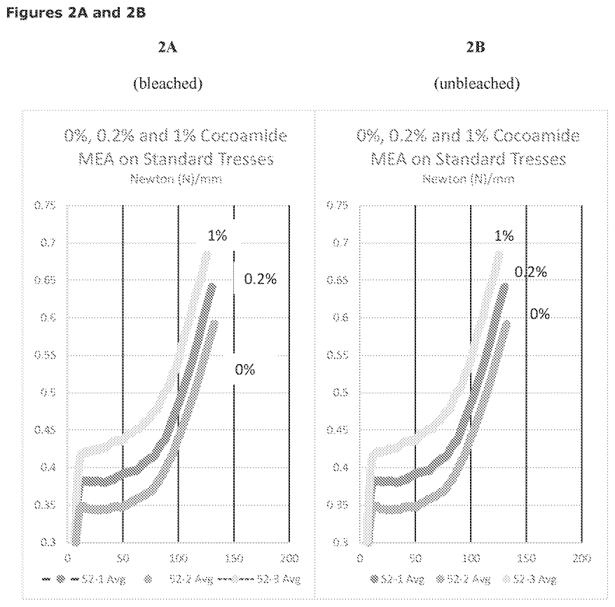

Surfactant concentration can enhance wetting and monomer absorption as demonstrated in FIGS. 2A and 2B.

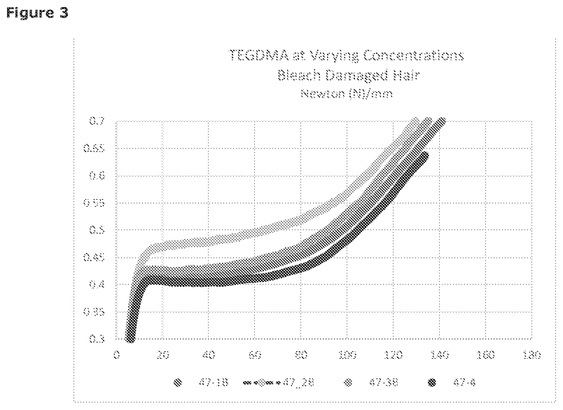

The oligomers and/or polymer chains that form inside the hair shaft are further reinforced by use of di-functional and/or tri-functional cross-linking agents to create three dimensional structures. Cross-linked structures forming inside the hair shaft may resemble miniature ropes, ladders, nets, and webs. While one or more of these structures may be formed in practice so some degree, it is hypothesized that polymerization inside the hair shaft, will lead to the formation of net or web-like structures that permeate throughout the hair shaft to reinforce and strengthen the entire structure from within. Increasing the amount of cross-linking improves the strength of the resulting polymer and the hair, as demonstrated in FIG. 3. Formation of a net-like structure prevents the formed polymers from completely sealing the hair to block/prevent migration of other hair products into or out of the hair shaft. Sealing the hair is less desirable because it would potentially block or hinder penetration of other hair products and prevent their use, e.g., hair color/lightening compositions, etc. For example, for on-head embodiments, the compositions do not block or hinder absorption of hair color or hair bleaching/lighting agents and these compositions may be used immediately before, during or after these types of common salon services.

(Meth)acrylate monomers with two or three reactive functional groups are useful and represent a preferred embodiment as cross-linking agents in the compositions, while monomers with four or more reactive functional groups are less preferred, primarily due to their higher molecular weight and more complex chain branching which typically create molecular structures too bulky for quick hair penetration, thereby rendering these higher functionality monomers as less preferred candidates for efficient hair absorption.

Efficient hair absorption is dependent on many factors, one of the most important of which is the size, shape, and polarity of the (meth)acrylate monomer molecule. For on-head embodiments, temperature is less of a factor, because the compositions are generally applied at ambient temperatures. In off-head embodiments, increasing the temperature of the composition and/or hair can be used to increase absorption rates and possibly lead to absorption of increased numbers of reactive monomers inside the hair shaft to further enhance their physical properties. UV, visible and polychromatic cured compositions are not polymerized by the heat created by a hair drying or straightening device allowing for the use of external heat sources suitable for use to drying, treat or process hair to facilitate increased absorption of the composition.

Thermally cured compositions are polymerized by the heat created by a hair drying or straightening device allowing for the use of external heat sources suitable for use to drying, treat or process hair to facilitate increased absorption of the composition, including IR, LED, or UV or polychromatic or full spectrum sources which create heat.

Different types of (meth)acrylate monomers and blends thereof produce (meth)acrylate oligomers and polymers (e.g., copolymers or terpolymers) that possess the physical properties which reflect the properties of the parent monomers. Many of the oligomer and/or polymer properties are determined almost exclusively by the structure and composition of the selected monomers. For instance, a water-soluble monomer would not be expected to polymerize to form a water-repellent polymer. In fact, the result could be a polymer with the ability to absorb water, and the reverse is also true.

Although not required for hair penetration and subsequent polymerization, cross-linking the (meth)acrylate monomers are known to increase the average molecular weight of the resulting oligomers and polymers inside the hair shaft and to enhance the physical properties of the oligomers and/or polymers. Varying the concentrations of cross-linking monomers also affects the final properties of the resulting polymer and of the hair as demonstrated in FIG. 3. Experiments show that improved physical properties of the treated hair are obtained when crosslinking monomers are used in the composition in a range between 0.1 and 25%, such as 0.1 to 10%, such as 1 to 20%, such as 3 to 15%, such as 5 to 15%, depending on the specific cross-linking monomer used.

Intact surfaces of hair with little to no visible damage are more difficult for the hair treatment composition to penetrate, while visibly damaged surfaces are easier to penetrate. The hair treatment compositions affect the outer cuticle layer of the hair in several important ways, such as by holding the cuticle layers in a smoother, flatter position, relative to the lengthwise axis of the hair shaft. The composition enters primarily by seeping between the eight to ten layers of cuticle on a typical hair shaft and polymerizes as a net-like structure inside the spaces or voids found within this area, thus beneficially slowing or hindering subsequent separation or preventing re-separation of these layers. Upon polymerization, the resulting oligomer and/or polymers reinforce these weak points by keeping them more intact and thus more resistant to deformation. Lifting, warping, and bending of the cuticle is a main cause of dull hair that feels rough and tangles easily. By using the hair treatment compositions described herein, these problems are resolved, and the treated hair regains a long-lasting smoother feel, increased shine, better manageability, and improved physical appearance.

The reactive monomers should be removed from the surface of the hair before mass polymerization occurs to prevent a residual polymer coating from developing on the outside of the hair upon drying. Also, the use of a heating source may exacerbate outer coating formation if excessive residual monomer remains on the hair surface. Thermal or photopolymerization on the outside of the hair shaft can also result in the formation of a sticky outer layer with negative sensorial properties. Any method/technique for use of the hair treatment compositions should ensure removal of reactive monomers prior to exposure to applied additional heat, UV, visible or polychromatic light sources sufficient luminous flux capable of initiating polymerization and/or before use of a hair dryer, flat-iron or other heat sources to prevent unwanted outer surface polymerization. Removal of residual reactive monomers on the hair surface is typically accomplished by first completely rinsing the hair with water until the water runs clean and then thoroughly washing the hair with shampoo.

Benefits associated with the present invention include 1) long-lasting or permanent repair of internal damage to the hair; 2) split ends are sealed and become resistant to re-splitting 3) added strength, durability, and increased protection of the treated hair from potential future harm; 4) improvements to treated hair that are cumulative and build with successive treatments; and 5) hair that is stronger, and exhibits improved physical condition, manageability, and appearance.

The present invention provides a practical, safe and effective method for the use of selected (meth)acrylate monomers, photoinitiators and specific wavelengths of UV, visible or polychromatic light and organic peroxides for the purpose of improving human hair, especially damaged hair, as well as an effective means of inducing and controlling thermal and/or photopolymerization inside the hair shaft, while preventing unwanted thermal and/or photopolymerization on the outer, exposed surface of the hair.

### Active Agents/Thermal and Photopolymerizable Monomers

The reactivity of the hair treatment compositions described herein is based primarily upon thermal or photopolymerizable acrylate and methacrylate monomers, such as acrylic and methacrylic acid esters. Acrylate monomers have the advantage of thermal or photopolymerizing more rapidly and more easily relative to their methacrylate counterparts which react more slowly. However, methacrylates are generally less irritating to skin compared to acrylates. In an embodiment of the present invention, uncontrolled rapid polymerization is undesirable for the reasons described herein. As a result, in some on-head embodiments, methacrylate monomers are the preferred choice over the acrylate monomers due to their lower potential for adverse skin reactions such as irritations or allergies, while acrylate monomers are useful for off-head hair applications.

Either methacrylate or acrylate monomers, or combinations thereof, may be used to treat human hair that has been previously cut, shaved, trimmed, or otherwise removed from the head, because such removal eliminates the potential for skin/scalp contact during treatment.

Suitable monofunctional methacrylate monomers, many with acrylate counterparts, include, but are not limited to, 1,2-dimethylpropyl methacrylate, 10-methylundecyl methacrylate, 11-methyldodecyl methacrylate, 12-methyltridecyl methacrylate, 13-methyltetradecyl methacrylate, 14-methylpentadecyl methacrylate, 15-methylhexadecyl methacrylate, 16-methylheptadecyl methacrylate, 2,3-epoxybutyl methacrylate, 2-diethylaminoethyl methacrylate, 2-ethoxyethyl methacrylate, 2-ethylhexyl methacrylate, 2-hydroxypropyl methacrylate, 2-isocyanatoethyl methacrylate, 2-methylbutyl methacrylate, 3,4-epoxybutyl methacrylate, 3-methylpentyl methacrylate, 4,5-epoxypentyl methacrylate, 4-methylpentyl methacrylate, 5-methylhexyl methacrylate, 6-methylheptyl methacrylate, 7-methyloctyl methacrylate, 8-methylnonyl methacrylate, 9-methyldecyl (meth)acrylate, acetoacetoxy ethyl methacrylate, butyl methacrylate, cetyl methacrylate, decyl methacrylate, dodecyl methacrylate, eicosyl methacrylate, ethyl methacrylate, ethylene glycol methacrylate, fluorinated methacrylate, glycidyl methacrylate, heneicosyl methacrylate, hentriacontyl methacrylate, heptacosyl methacrylate, heptadecyl methacrylate, heptyl methacrylate, hexacosyl methacrylate, hexadecyl methacrylate, hexadecyl methacrylate, hexyl methacrylate, hydroxy ethyl methacrylate, hydroxy propyl methacrylate, isobornyl cyclohexyl methacrylate, isobornyl methacrylate, isooctyl methacrylate, lauryl methacrylate, methoxy ethyl methacrylate, methoxy polyalkylene glycol methacrylate, methyl methacrylate, nonacosyl methacrylate, nonadecyl methacrylate, nonyl methacrylate, octacosyl methacrylate, octadecyl methacrylate, octadecyl methacrylate, octyl methacrylate, pentacosyl methacrylate, PEG-methacrylates, pentadecyl methacrylate, pentaerythritol methacrylate, pentyl methacrylate, polyglycerin methacrylate, stearyl methacrylate, t-butyl aminoethyl methacrylate, t-butyl methacrylate, tetracosyl methacrylate, tetradecyl methacrylate, t-octyl methacrylate, triacontyl methacrylate, triazine-2,4,6-tris(2-hydroxyethyl)tris methacrylate, tricosyl methacrylate, tridecyl methacrylate, undecyl methacrylate, and other commercially available monofunctional methacrylate monomers.

Suitable monofunctional acrylate monomers, include, but are not limited to, 1,2-dimethylpropyl acrylate, 10-methylundecyl acrylate, 11-methyldodecyl acrylate, 12-methyltridecyl acrylate, 13-methyltetradecyl acrylate, 14-methylpentadecylacrylate, 15-methylhexadecyl acrylate, 16-methylheptadecyl acrylate,2,3-epoxybutyl acrylate, 2-diethylaminoethyl acrylate, 2-ethoxyethyl acrylate, 2-ethylhexyl acrylate, 2-hydroxypropyl acrylate, 2-isocyanatoethyl acrylate, 2-methylbutyl acrylate, 3,4-epoxybutyl acrylate, 3-methylpentyl acrylate, 4,5-epoxypentyl acrylate, 4-methylpentyl acrylate, 5-methylhexyl acrylate, 6-methylheptyl acrylate, 7-methyloctyl acrylate, 8-methylnonyl acrylate, 9-methyldecyl acrylate, acetoacetoxy ethyl acrylate, butyl acrylate, cetyl acrylate, decyl acrylate, dodecyl acrylate, eicosyl acrylate, ethyl acrylate, ethylene glycol acrylate, fluorinated acrylate, glycidyl acrylate, heneicosyl acrylate, hentriacontyl acrylate, heptacosyl acrylate, heptadecyl acrylate, heptyl acrylate, hexacosyl acrylate, hexadecyl acrylate, hexadecyl acrylate, hexyl acrylate, hydroxy ethyl acrylate, hydroxy propyl acrylate, isobornyl cyclohexyl acrylate, isobornyl acrylate, isooctyl methacrylate, lauryl methacrylate, methoxy ethyl methacrylate, methoxy polyalkylene glycol acrylate, methyl acrylate, nonacosyl acrylate, nonadecyl acrylate, nonyl acrylate, octacosyl acrylate, octadecyl acrylate, octadecyl acrylate, octyl acrylate, pentacosyl acrylate, PEG-acrylates, pentadecyl acrylate, pentaerythritol acrylate, pentyl acrylate, polyglycerin acrylate, stearyl acrylate, t-butyl aminoethyl acrylate, t-butyl acrylate, tetracosyl acrylate, tetradecyl acrylate, t-octyl acrylate, triacontyl acrylate, triazine-2,4,6-tris(2-hydroxyethyl)tris acrylate, tricosyl acrylate, tridecyl acrylate, undecyl acrylate and other commercially available monofunctional acrylate monomers. In a preferred embodiment, the monofunctional acrylate monomer is cetyl acrylate, stearyl acrylate and lauryl acrylate.

Suitable difunctional and trifunctional (meth)acrylate monomers, include but are not limited to, polyethylene glycol dimethacrylate, polyethylene glycol diacrylate, 1,6-hexanediol diacrylate, 1,6-hexanediol dimethacrylate, 1,9-nonanediol dimethacrylate, 1,9-nonanediol diacrylate, 1,4-butanediol dimethacrylate, 1,4-butanediol diacrylate, 1,3-butanediol dimethacrylate, 1,3-butanediol diacrylate, 1, 12-dodecandiol dimethacrylate, 1,12-dodecandiol diacrylate, 1,10-decanediol dimethacrylate, 1,10-decanediol diacrylate, diurethane dimethacrylate, diurethane diacrylate, ethylene glycol dimethacrylate, ethylene glycol diacrylate, tetraethylene glycol dimethacrylate, tetraethylene glycol diacrylate, 1,5-pentanediol dimethacrylate, 1,5-pentanediol diacrylate, 1,4-phenylene diacrylate, 1,4-phenylene dimethacrylate, pentaerythritol methacrylate, pentaerythritol acrylate, polyethylene glycol dimethacrylate, polyethylene glycol diacrylate, diethylene glycol dimethacrylate, diethylene glycol diacrylate, triethylene glycol dimethacrylate (TEGDMA), triethylene glycol diacrylate, pentaerythritol dimethacrylate, pentaerythritol diacrylate, trimethylolpropane trimethacrylate, trimethylolpropane triacrylate, propoxylated (6) trimethylolpropane triacrylate, propoxylated (6) trimethylolpropane trimethacrylate, propoxylated glycerol triacrylate, propoxylated glycerol trimethacrylate, trimethylolpropane (EO) 9 trimethacrylate, trimethylolpropane (EO) 9 triacrylate, tricylcodecanol methacrylate, tricylcodecanol acrylate, glycerol trimethacrylate, glycerol triacrylate, pentaerythritol trimethacrylate, pentaerythritol triacrylate, ethoxylated glycerine trimethacrylate, ethoxylated glycerine triacrylate, trimethylolpropane trimethacrylate, trimethylolpropane triacrylate, 2,2-bis[4-(2′-hydroxy-3′-methacryloyloxy-propoxy)phenyl]propane, 2,2-bis[4-(2′-hydroxy-3′-acryloyloxy-propoxy)phenyl]propane, 1,1,1-tris[4-(2′-hydroxy-3′-methacryloyloxypropoxy)phenyl]ethane, 1,1,1-tris[4-(2′-hydroxy-3′-acryloyloxypropoxy)phenyl]ethane, tris[4-(2′-hydroxy-3′-methacryloyloxypropoxy)phenyl]methane, tris[4-(2′-hydroxy-3′-acryloyloxypropoxy)phenyl]methane and many commercially available difunctional and trifunctional monomers.

Difunctional and/or trifunctional (meth)acrylates are utilized to increase reactivity by increasing the number of reaction sites available per molecule within the narrow confines located inside a human hair and which act to increase internal cohesion of the oligomers/polymers formed inside the hair shaft and to dramatically increase the molecular weights of these monomers as previously described and also, facilitate chain branching and crosslinking.

In an exemplary embodiment, the hair treatment compositions are applied on-head or to hair on a human head. Several monomeric (meth)acylates are not particularly suitable for direct skin/scalp contact due to their potential to elicit adverse skin reactions with prolonged and/or repeated skin contact. While not desirable, inadvertent skin or scalp contact may occur. To help ensure skin and scalp safety, the preferred (meth)acrylate monomers are those with an inherently lower potential for eliciting adverse skin reactions, of which some non-limiting examples include mono-, di- and tri-functional methacrylate monomers ranging from lauryl (C12) methacrylate, tridecyl (C13) methacrylate, myristyl (C14) methacrylate, pentadecyl (C15) methacrylate, cetyl (C16) methacrylate, palmitic (C16) methacrylate, heptadecyl (C17) methacrylate, stearyl (C18) methacrylate, nonadecyl (C19) methacrylate, arachidic (C20) methacrylate, heneicosylic (C21) methacrylate, behenic (C22) fatty acid and tricosylic (C23) fatty acid which are classified structurally as fatty alcohol methacrylates (FAMA). FAMA which contain branching (such as one or more substituents off the carbon chain that include C₁₋₁₀ alkyl, OH, NH₂, NH(C₁₋₆ alkyl), SH, S(C₁₋₆ alkyl) and halogen (F, Cl, Br)) and/or one or more unsaturated (C═C or C≡C) bonds along with carbon chain are also suitable monomers for use in the hair treatment compositions of the invention. Their low potential to elicit adverse skin reactions is due to their relatively large molecular size, as well as being compositionally based on natural plant materials that are generally regarded as safe.

Other similar non-limiting examples of (meth)acylate monomers useful for human on-head applications that also exhibit an inherently lower potential for eliciting adverse skin reactions include certain mono-, di- and tri-functional ethoxylated or propoxylated monomers, such as those (meth)acrylates containing ethoxylated or propoxylated backbone structures. Some preferred examples are di-, tri- and tetra-ethylene glycol dimethacrylate. Some non-limiting examples of propoxylated monomers include propoxylated glycerol triacrylate, propoxylated (3)-trimethylolpropane triacrylate, propoxylated (6) trimethylolpropane triacrylate, and propoxylated (3)-glycerol triacrylate. These ethoxylated and propoxylated monomers offer lower skin irritation. Other suitable monomers include, but are not limited to, 2-(methacryloyloxy) ethyl acetoacetate and other Lonzamon™ monomers such as 2-(acetoacetoxy)ethyl methacrylate (AAEMA).

### Polymerization Initiators

A range of different chemical species exist that may be used to initiate the (meth)acylate polymerization with organic peroxides. Preferred initiators exhibit high reactivity, well documented safety, ease of use, wide availability, good shelf-life, and low cost. In an exemplary embodiment, the amount of the polymerization initiator ranges from between 0 and up to 5% by weight, such as 0.1 to 5% by weight, such as 0.5% to 5% by weight, such as 0.5% to 3% by weight, such as 0.7% to 3% by weight, such as 0.8% to 3% by weight, such as 0.5% to 2% by weight, such as 0.8% to 2% by weight. Some (meth)acrylates can be polymerized at elevated temperatures without the need for a free radical initiator, e.g., through the use of a heated flat iron which can reach temperatures of up to 450F/232C. However, heat induced monomer polymerization by itself (i.e., in the absence of an initiator) is typically a relatively slow process. It is preferable to accelerate such reactions and to maintain lower reaction temperatures by the addition of initiators so that polymerization is facilitated in the absence of the potentially damaging effects of high temperatures. Polymerization acceleration is also useful for overcoming oxygen inhibition of polymerization. One major drawback of using (meth)acrylate monomers is their high sensitivity towards oxygen which can inhibit and even block polymerization, particularly inside the cuticle layers near the air-monomer interface. Deeper inside the hair shaft, within the cortex, polymerization occurs more readily due to the absence of low levels of oxygen. To overcome inhibition in the cuticle layers, the reaction rate of the (meth)acrylic monomers is accelerated, causing polymerization to proceed faster than oxygen inhibition can prevent it. This increased rate of polymerization can be accomplished by employing suitable polymerization initiators and by use of an appropriate polymerization catalyst.

UV, visible or polychromatic light induced monomer photopolymerization products are self-polymerizing. In an embodiment, UV, visible and/or polychromatic photopolymerization is preferable over conventional thermal polymerization because such reactions can occur rapidly at room temperature with the addition of a photoinitiator and/or a synergist (if required) so that polymerization is facilitated in the absence of the potentially damaging effects of high temperatures often required by thermal initiators. UV, visible and polychromatic light polymerization is also useful for overcoming the oxygen inhibition requirements often associated with polymerization. One major drawback of using (meth)acrylate monomers is their high sensitivity towards oxygen, the presence of which can inhibit and even block polymerization, particularly inside the cuticle layers near the air-monomer interface deep inside the hair shaft. Within the cortex of the hair shaft, polymerization occurs more readily due to the low levels of oxygen. To overcome inhibition of the polymerization of the uncured hair treatment compositions in the cuticle layers, the reaction rate of the (meth)acrylic monomers is accelerated by the use of photoinitiators, causing polymerization to proceed faster than oxygen inhibition can prevent it. The rate of UV induced photopolymerization is determined by varying the concentration by weight a suitable photopolymerization initiator in combination with exposure to the appropriate useful wavelengths with the correct intensities. A catalyst, synergist, or accelerator may also be used to help control the rate of polymerization and the depth of cure with difficult-to-cure substrates such as those which are highly colored with dyes and/or lake pigments (typically in metallic salt form) containing colorants.

Without being bound by any theory or hypothesis, the presently accepted mechanism for free radical polymerization for (meth)acrylates is as follows: the first step forms the free radical species (R·) which reacts with an acrylic monomer in a process called initiation. Typically, the free radical species attacks the less sterically hindered carbon atom of the (meth)acrylate molecule to form a more stable carbon centered radical which in turn attacks the double bond of a nearby (meth)acrylate monomer to form an energized dimer species which in turn reacts with another monomer to form an energized trimer, and so on. The well-known progressive addition of new monomers to the growing oligomer/polymer chain is termed chain propagation, creating a head-to-tail arrangement of monomers into chains.

Organic peroxides contain a weak peroxide bond (—O—O—) that when exposed to suitable conditions will symmetrically cleave the covalent bond located between the two oxygen atoms to yield two individual highly energetic species which are relatively stable carbon-centered radicals, For example, upon decomposition, benzoyl peroxide (PhC)OO)₂ yields two benzoyloxyl radicals (PhCOO·) which initiate the polymerization process. Non-limiting examples of organic peroxides include benzoyl peroxide, dicumyl peroxide, tert-butyl-peroxide, cumene hydroperoxide, diacetyl peroxide, acetyl acetone peroxide, ascaridole, di-(1-naphthoyl) peroxide and methyl ethyl ketone peroxide. A preferred organic peroxide initiator is benzoyl peroxide, also known as dibenzoyl peroxide, which consists of two benzoyl groups bridged by a peroxide (—O—O—) link. Benzoyl peroxide is preferred because it can be catalyzed to initiate polymerizations at ambient temperatures, as well as having a long history of safe use in cosmetic and consumer products.

Typically, aromatic or aliphatic tertiary amines are considered most suitable for catalyzing organic peroxides to initiate (meth)acrylate polymerization. Non limiting examples include, but are not limited to, N, N-dimethyl-p-toluidine, N,N-diethyl-p-toluidine, N, N-dimethyl aniline, N, N-bis(2-hydroxyethyl-p-toluidine), 4-(dimethylamino)phenethyl alcohol and ethyl 4-(dimethyl amino)benzoate.

### Catalysts/Synergists

Many different catalysts or synergists may be used in the hair treatment compositions, including amines (including aromatic amines), acylated amines, acrylated amines and acrylated amine oligomers. Typically, aromatic or aliphatic tertiary amines are considered most suitable for catalyzing organic peroxides to initiate (meth)acrylate polymerization. Suitable examples of catalysts or synergists include, but are not limited to, triethanolamine, 2-ethyl-4-(dimethylamino)benzoate, 4-benzoyl 4′-methyldiphenyl sulfide, 2-ethylhexyl 4-(dimethylamino)benzoate, 1-(ethoxycarbonyl)prop-1-yl dithiobenzoate, N,N-dimethyl-p-toluidine, N,N-diethyl-p-toluidine, N,N-dimethyl aniline, N, N-bis(2-hydroxyethyl-p-toluidine), and 4-(dimethylamino)phenethyl alcohol.

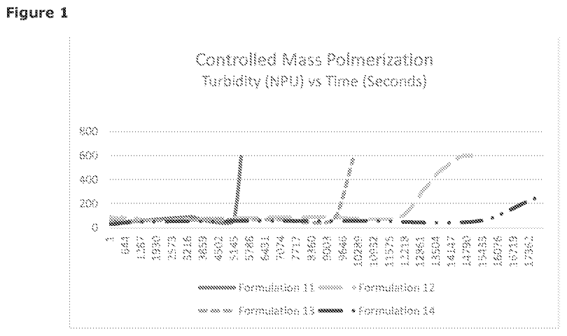

For embodiments where the hair treatment composition is applied on-head and a catalyst is used, a preferred catalyst is ethyl 4-(dimethylamino) benzoate because it is considered toxicologically acceptable and allows for suitably long induction periods before the on-set of mass polymerization while promoting rapid acceleration in the polymerization rate after the predetermined induction period has passed, characterized as the period before a rapid and significant acceleration in polymerization as demonstrated in FIG. 1. Compositions contain 0 to 2%, such as 0 to 1%, such as 0 to 0.50%, such as between 0 and up to 2%, such as 0.05% to 2%, such as 0.05% to 1.5%, such as 0.05% to 1%, such as 0.05% to 0.5%, such as 0.1% to 2%, such as 0.1% to 1%, weight of the polymerization catalyst.

### UV/Vis or Polychromatic Photopolymerization Initiators

Any type of Norrish Type I or Type II photoinitiator which, upon UV/Vis irradiation or polychromatic irradiation, forms free radicals can be employed in these novel compositions, including but not limited to benzoins and substituted benzoins, including benzoin, benzoin ether derivatives, such as benzoin methyl ether, benzoin ethyl ether and benzoin isopropyl ether, benzoin phenyl ether and benzoin acetate, acetophenones, including acetophenone, 2,2-dimethoxyacetophenone and 1,1-dichloroacetophenone, benzyl, benzyl formats, benzyl ketals, such as benzyl dimethyl ketal and benzyl diethyl ketal, anthraquinones, including 2-methylanthraquinone, 2-ethylanthraquinone, 2-tert-butylanthraquinone, 1-chloroanthraquinone and 2-amylanthraquinone, triphenylphosphine, acylphosphine oxides, amino acetophenones, benzoylphosphine oxides for example 2,4,6-trimethylbenzoyldiphenylphosphine oxide, benzophenones, such as benzophenone and 4,4′-bis(N,N′-dimethylamino)benzophenone, thioxanthones such as isopropyl thioxanthones and xanthones, acridine derivatives, phenazine derivatives, quinoxaline derivatives or 1-phenyl-1,2-propanedione, 2-O-benzoyl oxime, alpha amnio ketones and hydroxyalkyl ketones such as 1-aminophenyl ketones or 1-hydroxyphenyl ketones, such as 1-hydroxycyclohexyl phenyl ketone, phenyl 1-hydroxyisopropyl ketone and 4-isopropylphenyl 1-hydroxyisopropyl ketone, all of which are known compounds. Some non-limiting examples of UV/Vis photopolymerizable initiators are 2-isoproply thioxanthone aka Omirad ITX, or 2,2-dimethoxy-1,2-diphenylethan-1-one aka Omirad BDK, or 2-benzyl-2-(dimethylamino)-1-[4-(morpholinyl)phenyl)]-1-butanone aka Irgacure® 369, or bis (2,4,6, trimethylbenzoyl)-phenyphosphinate, aka Irgacure® 189, or 2-methyl-1 [4-methylthio)phenyl]-2-morpholinopropan-1-one aka Irgacure® 907, benzophenone, 2-dimethylamino-2-(4-methyl-benzyl)-1-(4-morpholin-4-yl-phenyl)-butan-1-one aka Irgacure® 379, or -1-hydroxycyclohexyl phenyl ketone aka Irgacure® 184, or -2-hydroxy-2-methyl-phenyl-propane-1-one aka Irgacure® 1173, or 1-[4-(2-hydroxyethoxy)-phenyl]-2-hydroxy-2-methyl-1-propane-1-one aka Irgacure® 2959, or 2,4,6-trimethylbenzoyldiphenylphosphine oxide aka Omirad TPO, or ethyl (2,4,6-trimethylbenzoyl) phenyl phosphinate aka Omirad TPO-L, or 2,2-dimethoxy-2-phenylacetophenone aka Irgacure® 651, and camphorquinone and benzophenone, all of which are known compounds.

For embodiments where the hair treatment composition is applied on-head, the preferred UV/Vis photoinitiator is TPO-L, aka ethyl (2,4,6-trimethylbenzoyl) phenyl phosphate, because it is considered toxicologically acceptable and allows for rapid UV induced photopolymerization with almost instant on-set of mass polymerization. Compositions contain 0 to 5%, such as 0 to 2%, such as 0 to 0.50%, such as between 0 and up to 5%, such as 0.05% to 5%, such as 0.05% to 1.5%, such as 0.05% to 2%, such as 0.05% to 0.5%, such as 0.1% to 5%, such as 0.1% to 2%, weight of the polymerization catalyst or synergist as demonstrated in Table 5.

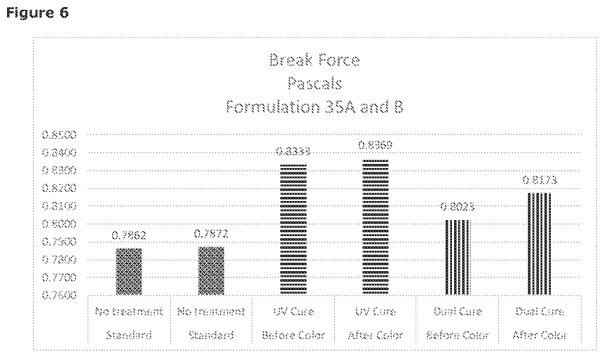

For embodiments where the hair treatment composition is applied on-head, the preferred UV/Vis photoinitiator is TPO-L, aka ethyl (2,4,6-trimethylbenzoyl) phenyl phosphate in combination with a BPO/Carrier powder dual curing system as demonstrated in FIG. 6 demonstrating that dual thermal/UV curing is achievable, also demonstrating that these treatments can be successfully applied immediately before or immediately following hair coloring treatments.

For embodiments where the hair treatment composition is applied on-head, the preferred UV/Vis photoinitiator is TPO-L, aka ethyl (2,4,6-trimethylbenzoyl) phenyl phosphate because it is considered toxicologically acceptable and allows for rapid UV induced photopolymerization with almost instant on-set of mass polymerization.

### Solid Carrier

In an exemplary embodiment, a two-part system is used to prevent polymerization of the monomers in the composition from occurring until the two parts are blended. This is especially useful for on-head applications. Part A contains the reactive monomer and optional polymerization catalyst, while Part B contains the polymerization initiator alone or in combination with a solid carrier, such as an inert solid carrier, such as an inert carrier powder. The initiator carrier can be any solid powder that is inert, which is defined as non-reactive with the polymerization initiator and does not interfere with normal monomer absorption into the hair shaft or subsequent polymerization processes. The carrier may include conventional natural organic powders, mineral powders, cellulose derivatives, synthetic powder, and polymer beads. Compositions contain 0 to 50%, such as 0 to 40%, such as 0 to 30%, such as 0 to 20%, such as 5% to 50%, such as 5% to 40%, such as 5% to 30%, such as 5% to 20%, such as 10% to 50%, such as 10% to 30%, such as 15% to 50%, such as 15% to 30%, by weight of the solid carrier. The solid carrier may contain small concentrations of water, but preferably do not add more than 5% by weight of water to the composition since excess water can slow or hinder the polymerization process.

Non-limiting examples of carrier powder additives include natural or synthetic mineral quartz such as silicon dioxide powders, preferably non-crystalline, amorphous silicon dioxide (also known as silica) and microcrystalline cellulose or blends thereof are preferred. Other non-limiting examples are magnesium aluminum silicate and inorganic clays such as calcium carbonate, calcium sulfate dihydrate, magnesium sulfate, kaolin, French green clay, rhasssoul clay, fuller's earth clay, red Moroccan clay, Rose clay, Dead sea clay, illite clay, acrylic powder and bentonite with particle sizes approximately and primarily ranging between 1 to 500 microns diameter, such as 5 to 400 microns, such as 5 to 250 microns, such as 5 to 100 microns, such as 10 to 100 microns, such as 15 to 80 microns.

### Liquid Carrier

In an exemplary embodiment, a two-part system is used to prevent polymerization of the monomers in the composition from occurring until after the two parts are blended. This is especially useful for on-head applications. Part A contains the reactive monomer and catalyst, while Part B is comprised of polymerization initiator in combination with an inert liquid. The carrier can be any liquid that is inert, which is defined as non-reactive with the polymerization initiator and does not significantly interfere with monomer absorption into the hair shaft or subsequent polymerization processes. Compositions contain 0 to 50%, such as 0 to 40%, such as 0 to 30%, such as 0 to 20%, such as 5% to 50%, such as 5% to 40%, such as 5% to 30%, such as 5% to 20%, such as 10% to 50%, such as 10% to 30%, such as 15% to 50%, such as 15% to 30%, by weight of the liquid carrier. In one example, the preferred carrier is water, however the composition does not add more than 5% by weight of water due to the carrier, such as no more than 2% by weight, such as no more than 1% by weight, or polymerization can be adversely affected. Preferably, the water concentration does not exceed 1% by weight.

### Solvents

In an exemplary embodiment, the liquid hair treatment compositions of the present invention are essentially water-free and considered lipophilic in nature. In another embodiment, a volatile organic solvent is used to lower the viscosity of the composition to enhance rates of absorption of the composition into the hair shaft and to solubilize surface oils and to facilitate removal of additional water by leaching it from inside the hair shaft allowing for replacement by monomers. This leaching property is important because water can compete with the monomers for space inside the hair strand. For this reason, the amount of water in the hair treatment composition should be less than 5% by weight, such as less than 3%, such as less than 1%, such as less than 0.5%, such as 0%. Typically, the total volatile organic solvent (VOC) concentration ranges from 0 to 40% by weight, such as 0 to 35%, such as 0 to 25%, such as 0 to 10%, such as 0 to 5%, such as 0 to 2%, such as 1% to 40% by weight, such as 1% to 30%, such as 1% to 20%, such as 1% to 10%, such as 5% to 40%, such as 5% to 25%, such as 5% to 15%, such as 10% to 40%, such as 10% to 30%, and therefore can be formulated to be compliant with any applicable VOC regulations. Some non-limiting examples of volatile organic solvents include, but are not limited to, one or more of acetone, ethanol, butanol, isopropanol, methanol, methyl acetate, ethyl acetate, methyl ethyl ketone, ethyl ether, isobutyl ketone, etc.

In an exemplary on-head treatment, ethanol is the volatile organic solvent utilized in the hair treatment composition. In a particular embodiment, the ethanol is denatured 200 proof absolute ethanol that is essentially water-free.

### Surfactants and Penetration Enhancers

Surface active agents, also known as surfactants, may be used to assist in the solubilizing and removal of oils and other debris that could physically block monomer absorption into the hair shaft. Suitable surfactants enhance absorption by improved wetting and removal of hindering surface oils or other debris without adversely impacting the polymerization process. Surfactants are selected based on their ability to remove absorption hindering substances from the surface of the hair, allowing for improved penetration, and enhancing physical properties as a result, as demonstrated in FIG. 2.

Surfactants also facilitate the removal of reactive monomers from the outer surface of the hair shaft, thereby reducing the presence of residual monomers and lessening the potential for undesired sticky polymer formation on the outer surface of the hair strand. Many types of surfactants are commercially available, and any that are soluble in the hair treatment compositions may be used for the stated purposes. Non-limiting examples of suitable surfactants include, but are not limited to, triglyceride ethoxylates and sorbitan surfactants (such as sorbitan oleate, sorbitan olivate) or ethoxylated fatty acids (such as (POE (20) sorbitan monolaurate, sorbitan monostearate, sorbitan tristearate), as well as, alkoxylated amides (such as PEG-7 oleamide, PEG-9 oleamide, PEG-4 rapeseed amide, PEG-4 stearamide, PEG-10 stearamide, PEG-15 stearamide, PEG-5 tallow amide, PEG-8 tallow amide, PEG-2 tallow amide DEA, polyglyceryl-4-PEG-2 cocamide, PPG-2 cocamide, PPG-1 hydroxyethyl capryl amide, PPG-2 hydroxyethyl cocamide, PPG-2 hydroxyethyl coco/isostearamide, PEG-6 hydrogenated palm amide, PEG-13 hydrogenated tallow amide, PEG-5 lanolin amide, PEG-3 lauramide, PEG-2 lauramide and PEG-3 oleamide).

Preferred examples of surfactants include cocoamide MEA, PPG-2 cocamide, PPG-2 hydroxyethyl coco/isostearamide, PPG-2 hydroxyethyl cocamide and POE (20) sorbitan monolaurate.

Typically, surfactants are present in amounts of 0 to 15% by weight, such as 0 to 10%, such as 0 to 5%, such as 1% to 10%, such as 1% to 5%, such as 5% to 15%, such as 5% to 10%.

Specific lipophilic penetration enhancing substances known to facilitate keratin absorption may also be utilized in some embodiments, especially in on-head applications where faster penetration rates are desirable. While not being bound by any theory or hypothesis, it is believed that the penetration enhancers lower inherent barrier properties of the cuticle by transiently loosening the tight junction between adjacent cells to increase membrane fluidity and do so without inducing the hair damage seen by agents that reduce disulfide bonds.

Examples of suitable lipophilic penetration enhancers include, but are not limited to, fatty acid alcohols, alpha-bisabolol, dimethyl isosorbide, ethoxydiglycol, phosolipids, ethanolic substances, azone, dimethyl sulfoxide, isopropyl myristate, dimethyl 9-decenamide and some essential oils such as lavender, tea tree, eucalyptus, black cumin, wintergreen, camphor, tetrahydropiperine, alpha-lipoic acid, oleic acid and palmitic acid. For on-head embodiments, fatty acid alcohols such as oleyl alcohol, essential oils such as lavender, dimethyl isosorbide and ethanolic substances are preferred. Typically, individual penetration enhancers are used in amounts of 0 to 20% by weight, such as 0 to 15%, such as 1% to 10%, such as 1% to 5%, such as 5 to 15%, such as 5% to 10%.

### Alkalizing Agents/pH Adjusters

Suitable alkalizing agents and pH adjusters include, but are not limited to, monoethanolamine, diethanolamine, triethanolamine, aminomethyl propane, aminomethyl propanediol, 2-amino-2-methyl-1,3-propanediol, diamino methylpropane, tromethamine, tetrahydroxypropyl ethylenediamine and dimethyl aminomethyl propanol.

Typically, alkalizing agents and pH adjusters are used in amounts of 0 to 5% by weight, such as 0 to 3%, such as 1% to 5%, such as 1% to 3%, such as 2% to 5%.

The pH values listed herein are determined from an aqueous blend that consists of 50% water and 50% of the particular hair treatment composition being measured.

Unless stated otherwise, all pH measurements were conducted using a Mettler-Toledo SevenMulti PH meter and an InLab Viscous Pro ISM pH probe calibrated at four separate pH values with Mettler-Toledo pH standards.

As an example of pH values associated with an exemplary hair treatment composition in the absence of an alkalizing agent, the composition containing cetyl methacrylate and cetyl acrylate was tested. The composition was measured neat (since it contained as an ingredient 1% or 2% by weight of a blend of 75% BPO/25% water) and as a 50:50 blend of composition to water. The results are shown below.

| | | pH 50:50 |
|---|---|---|
| Formula | pH neat | DI water |
| Cetyl methacrylate + 75% BPO/ | 4.4 | 3.8 |
| Water @ 2% | | |
| Cetyl acrylate + 75% BPO/Water @ 2% | 3.8 | 4.7 |
| Cetyl methacrylate + 75% BPO/ | 4.3 | 4.1 |
| Water @ 1% | | |
| Cetyl acrylate + 75% BPO/Water @ 1% | 3.5 | 4.3 |

As an example of pH values associated with an exemplary hair treatment composition in the absence of an alkalizing agent, the composition containing cetyl methacrylate and cetyl acrylate was tested. The composition was measured neat as a blend of cetyl methacrylate or cetyl acrylate with 5% and 1% TPO-L or 5% and 1% Camphorquinone) and as a 50:50 blend of composition to water. The water-free neat and with 50% distilled water results are shown below.

| Formula | pH neat | pH 50:50 DI water |
|---|---|---|
| Cetyl methacrylate/5% TPO-L | 4.5 | 4.0 |
| Cetyl acrylate/5% TPO-L | 3.7 | 4.5 |
| Cetyl methacrylate/1% TPO-L | 4.4 | 4.2 |
| Cetyl acrylate1% TPO-L | 4.4 | 4.2 |

### Colorants and Optical Brighteners

Any conventional soluble colorant or optical brightener can be used if it does not interfere with absorption or subsequent polymerization. A preferred colorant or optical brightener has a molecular weight below 500 Daltons to facilitate hair absorption. Suitable colorants include, but are not limited to, color additives that are subject to certification and permitted by the Food and Drug Administration (FDA) for use in cosmetics, as described in 21 Code of Federal Regulations (CFR) Part 74, subparts C-Cosmetics; 21 CFR Part 82, subparts B, C and D; and 21 CFR Part 73, subpart C-Cosmetics and/or those listed by the European Commission in the European Cosmetic Ingredient (“Cosing”) Database. Suitable optical brighteners, also known as fluorescent brighteners, include materials which absorb light in the ultraviolet region and re-emit light in the blue region by fluorescence, typically in the 420-460 nm range. Non-limiting examples are selected from the group consisting of trans-stilbene, coumarin, pyrazine, diazoles, triazoles, oxazoles, porphyrins, triazines, imidazoles, and mixtures thereof and include C.I. Fluorescent Brightener 61 (BOC Sciences) and Fluorescent Brightener 184 also known as Uvitex® OB (BASF).

Typically, colorants and optical brighteners are used in amounts of 0 to 5% by weight, such as 0 to 3%, such as 1% to 5%, such as 1% to 3%, such as 2% to 5%.

### Conditioning Agents

Cosmetic hair conditioning agents known to those skilled in the arts may be added to the composition to enhance the final appearance and condition of the hair if they are soluble in the other ingredients, do not react with other ingredients, do not prevent absorption into the hair and do not prevent polymerization inside the hair. Non-limiting examples include selected fatty acids and their esters, fatty alcohols, fatty amines, fatty amides, such as cetyl alcohol, phytantriol, oleylamine, oleamide, omega-3-fatty acid and its methyl ester.

Typically, hair conditioning agents are used in amounts of 0 to 5% by weight, such as 0 to 3%, such as 1% to 5%, such as 1% to 3%, such as 2% to 5%.

### Exemplary Compositions

An exemplary heat curing hair treatment composition of the invention with suitable amounts of each of the listed components is shown below.

| Ingredient | % by weight |
|---|---|
| (meth)acrylate monomer(s)¹ | 10 to 99 |
| di(meth)acrylate monomer(s)¹ | 0 to 20 |
| tri(meth)acrylate monomer(s)¹ | 0 to 5 |
| heat activated polymerization initiator | between 0 and up to 5 |
| polymerization catalyst | 0 to 2 |
| surfactant | 0 to 15 |
| penetration enhancer | 0 to 20 |
| pH adjuster/alkalizing agent | 0 to 5 |
| carrier | 0 to 50 |
| organic solvent | 0 to 40 |
| water | 0 to <5 |
| colorant/optical brightener | 0 to 5 |
| conditioning agent | 0 to 5 |

¹total amount of the (meth)acrylate, di(meth)acrylate, and tri(meth)acrylate monomers = least 20% by weight of the total composition

### Exemplary UV or Visible or Polychromatic Light Curing Compositions

An exemplary UV or visible or polychromatic light curing hair treatment composition of the invention with suitable amounts of each of the listed components is shown below.

| Ingredient | % by weight |
|---|---|
| (meth)acrylate monomer(s)¹ | 10 to 99 |
| di(meth)acrylate monomer(s)¹ | 0 to 20 |
| tri(meth)acrylate monomer(s)¹ | 0 to 5 |
| UV/Vis polymerization initiator | between 0 and up to 5 |
| polymerization catalyst or synergist | 0 to 2 |
| surfactant | 0 to 15 |
| penetration enhancer | 0 to 20 |
| pH adjuster/alkalizing agent | 0 to 5 |
| organic solvent | 0 to 80 |
| colorant/optical brightener | 0 to 5 |
| conditioning agent | 0 to 5 |

¹total amount of the (meth)acrylate, di(meth)acrylate and tri(meth)acrylate monomers = least 20% by weight of the total composition.

### Experimental Test Methods and Results

Formulations were prepared for testing and are identified below.

Stress/strain testing was conducted using either a Shimadzu AGS-JSTD or an AGX-S “AUTOGRAPH” series tabletop universal tester (commercially available from Shimadzu Scientific Instruments; Columbia, MD) equipped with a 50 N load cell. Single hair strands with a gauge length of 150 mm were mounted to cardboard tabs with ethyl cyanoacrylate glue (Gorilla Super Glue; The Gorilla Super Glue Company; Cincinnati, OH). The cardboard tabs were secured to the machine grips and tested with a crosshead speed of 25 mm/min. The results shown are the observed averages of 15-30 stress/strain tests on individual hairs tested using 250 mm (10 in) hair tresses, both bleached and unbleached when treated in a single application with an exemplary hair treatment composition of the invention.

Table 1 demonstrates the improvement in strength observed in both treated high porosity and treated low porosity hair using Formulation 7 compared to the hair prior to treatment. More specifically, the results of the stress/strain tests conducted on a bleached hair tress and an unbleached hair tress (a natural, brown European tress) using Formulation 7 as shown in Table 1 demonstrates an improved energy-to-break increase of 14.6% and 13.7%, respectively. These results are evidence that even hair which appears to be in healthy condition experiences a significant improvement in measured physical properties when treated with a hair treatment composition of the present invention.

Table 2 demonstrates that repeated washing of the hair does not diminish the improvements to the hair resulting from treatment with an exemplary hair treatment composition of the invention (Formulation 7) and that there are cumulative benefits to the hair observed after a second treatment.

More specifically, hair tress samples previously treated with the hair treatment composition of Formulation 7 were tested after one treatment (#1) and tested again after being shampooed eight times on alternating days for 16 days (#2), and then retreated with the same composition and retested. The results in Table 2 demonstrate an additional 10% increase in the energy required to break the hair after the second treatment (#3), demonstrating that treatments with a hair treatment composition of the invention can be cumulative, thus further improving the physical properties of hair by additional treatments to better protect it from future harm or damage.

Tables 3A and 3B show the results of stress/strain testing of wet versus dry treated bleached hair compared to untreated bleached hair.

The Elastic Modulus (EM) and Energy-to-Break (EB) properties were measured for single strands of bleached hair treated with the same composition and applied to both wet (“Wet”) and dry (“Dry”) hair. These samples were then each compared to an untreated bleached hair standard (“Std”) that was measured in the identical fashion. This testing demonstrates a 13.5% (dry) and 15.5% (wet) increase in Elastic Modulus, as well as a 14.6% increase in Energy-to-Break for both dry and wet samples.

Table 4 shows that co-monomers can increase the energy required to break a single strand of hair. This non-limiting study demonstrates how increasing the concentration of a comonomer while decreasing the concentration of the primary monomer produced significant increases in the Energy-to-Break values obtained by stress/strain testing. The tresses were treated when the compositions contained various concentrations of the monofunctional hexadecyl methacrylate (also known as cetyl methacrylate) and acetoacetoxyethyl methacrylate (AAEMA). Table 5 demonstrates that increasing the concentration of photoinitiator produces a significant increase in the Break Force values obtained by stress/strain testing.

Methodology: The onset of polymerization was determined by measuring changes in the turbidity of the composition starting as a slightly hazy, water-white solution prior to polymerization and transforming into an opaque white solid. Turbidity changes were recorded once per second using a photometric turbidity meter. Turbidity was determined using Neulog Turbidity Sensors purchased from Carolina Biological Supply, Burlington, NC. The sensors were digitally interfaced with a standard personal computer using a Neulog USB Bridge module which transferred the collected data to the computer for tabulation and graphing. The only modification to these devices was to slightly widen the opening to the sensor chamber to allow for solvent-resistant precision quartz fluorometer cuvettes (Sigma-Aldrich) to be utilized. Blended samples were placed into the quartz cuvettes and mounted in the turbidity sensor. Turbidities were typically monitored, and changes recorded for eight to ten hours, and the results observed using the Neulog graphing software and duplicated using Excel. The results as shown in FIG. 1. The onset of mass polymerization was determined to be the point where the slope of the turbidity curve substantially deviates from the baseline.

FIG. 1 depicts the results of testing the exemplary hair treatment compositions of Formulations 11, 12, 13 and 14. Mass polymerization was delayed (induction period) for 5461 seconds (1.5 hours) for Formulation 11; 9203 seconds (2.6 hours) for Formulation 12; 11,992 seconds (3.4 hours) for Formulation 13; and 15,529 seconds (4.3 hours) for Formulation 14. The observed times represent the time it takes for each of the hair treatment compositions to begin polymerizing without the addition of external heat (e.g., blow dryers). Longer induction times allow the recipient's hair to absorb a higher amount of the product before oligomerization or full polymerization occurs inside the hair shaft.

FIGS. 2A and 2B shows that increasing the concentration of the surfactant Cocoamide MEA in the treatment of unbleached and bleached damaged hair as evidenced by comparing the results of tested Formulations 52-1, 52-2 and 52-3 increases absorption of each formulation inside the hair shaft which results in an increase in overall strength, where the formulations of 52-1, 52-2 and 52-3 are listed below in Formulations 15-17.

FIG. 3 demonstrates the effects of using formulations with increasing concentrations of the cross-linker triethylene glycol dimethacrylate (TEGDMA) on bleach damaged hair, Formulations 18-20.

FIG. 4 demonstrates that the ideal exposure time for this formulation is 30 minutes.

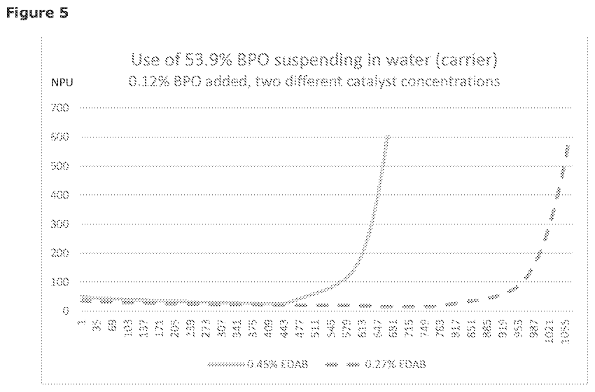

FIG. 5 demonstrates two different polymerization induction times obtained when water is used as an inert carrier for BPO, Formulation 23.

FIG. 6 demonstrates both UV curing and dual thermal/UV curing formulations can be successfully applied immediately before or immediately following hair coloring treatments to achieve similar results and that hair color services do not block absorption or hinder polymerization, Formulations 35A and 35B.

### Formulations

Formulation 1. Exemplary high alkalizer formulation with comonomer; generally preferable for non-porous/resistant hair.

| Ingredient | % by weight |
|---|---|
| Dimethyl Amino Methyl Propanol | 0.80 |
| Tetraethylene glycol dimethacrylate | 8.2 |
| 2-(Acetoacetoxy)ethyl methacrylate | 4.0 |
| Cetyl Methacrylate | 67.7 |
| Ethanol 200 pf. | 6.0 |
| Dimethyl Isosorbide | 1.0 |
| PPG-2 Hydroxyethyl Cocamide | 5.0 |
| Ethyl 4-(dimethyl amino) benzoate | 0.3 |
| Silica/15% BPO | 7.0 |

Formulation 2. Exemplary high alkalizer formulation with alternate monomer and surfactant, no co-monomer: generally preferable for resistant hair.

| Ingredient | % by weight |
|---|---|
| Dimethyl Amino Methyl Propanol | 1.05 |
| 1,12-dodecanediol dimethacrylate | 10.0 |
| Stearyl Methacrylate | 66.61 |
| Ethanol 200 pf. | 6.0 |
| Dimethyl Isosorbide | 1.04 |
| Cocamide MEA | 0.2 |
| Ethyl 4-(dimethyl amino) benzoate | 0.6 |
| Silica/9.46% BPO | 14.5 |

Formulation 3. Exemplary alkalizer-free, low pH formulation with comonomer: generally preferable for porous/non-resistant hair.

| Ingredient | % by weight |
|---|---|
| Tetraethylene glycol dimethacrylate | 8.0 |
| 2-(Acetoacetoxy)ethyl methacrylate | 4.0 |
| Cetyl Methacrylate | 65.55 |
| Ethanol/ 200 proof | 6.0 |
| Dimethyl Isosorbide | 1.0 |
| PPG-2 Hydroxyethyl Cocamide | 6.75 |
| Ethyl 4-(dimethyl amino) benzoate | 0.5 |
| Silica/15% BPO | 8.2 |

Formulation 4. Exemplary medium level alkalizer formulation generally preferable for low porosity/resistant hair.

| Ingredient | % by weight |
|---|---|
| Dimethyl Amino Methyl Propanol | 0.60 |
| Tetraethylene glycol dimethacrylate | 8.0 |
| 2-(Acetoacetoxy)ethyl methacrylate | 4.0 |
| Cetyl Methacrylate | 66.1 |
| Ethanol 200 pf. | 6.0 |
| Dimethyl Isosorbide | 1.0 |
| PPG-2 Hydroxyethyl Cocamide | 6.0 |
| Ethyl 4-(dimethyl amino) benzoate | 0.3 |
| Silica/15% BPO | 8.0 |

Formulation 5. Exemplary alkalizer-free formulation with alternate monomer and no co-monomer.

| Ingredient | % by weight |
|---|---|
| Tetraethylene glycol dimethacrylate | 7.0 |
| Stearyl Methacrylate | 71.25 |
| Ethanol 200 pf. | 6.0 |
| Dimethyl Isosorbide | 1.0 |
| PPG-2 Hydroxyethyl Cocamide | 5.5 |
| Ethyl 4-(dimethyl amino) benzoate | 0.25 |
| Silica/15% BPO | 9.0 |

Formulation 6. Exemplary medium level alkalizer formulation with no comonomer: generally, for all types of hair.

| Ingredient | % by weight |
|---|---|
| Dimethyl Amino Methyl Propanol | 0.50 |
| Cetyl Methacrylate | 76.1 |
| Ethanol 200 pf. | 7.0 |
| Dimethyl Isosorbide | 0.9 |
| Cocoamide MEA | 5.3 |
| Ethyl 4-(dimethyl amino) benzoate | 0.2 |
| Silica/15% BPO | 10.0 |

Formulation 7. Exemplary formulation for Table 1 and 2, high and low porosity hair, resisting wash out to provide cumulative benefits.

| Ingredient | % by weight |
|---|---|
| Dimethyl Amino Methyl Propanol | 0.9 |
| Tetraethylene glycol dimethacrylate | 8 |
| 2-(Acetoacetoxy)ethyl methacrylate) | 4 |
| Cetyl Methacrylate | 68.6 |
| Ethanol 200 pf. | 6 |
| Dimethyl Isosorbide | 1 |
| Cocamide MEA | 1 |
| PPG-2 Hydroxyethyl Cocamide. | 3 |
| Ethyl 4-(dimethyl amino) benzoate | 0.7 |
| Silica/13.3% BPO | 6.8 |

Formulations 8-10. Exemplary formulations with varying concentrations of co-monomer.

| | (8) | (9) | (10) |
|---|---|---|---|
| | % by | % by | % by |
| Ingredient | weight | weight | weight |
| Dimethyl Amino Methyl Propanol | 0.45 | 0.45 | 0.45 |
| Tetraethylene glycol | 8 | 8 | 8 |
| dimethacrylate | | | |
| 2-(Acetoacetoxy)ethyl | 2 | 6 | 8 |
| methacrylate) | | | |
| Cetyl Methacrylate | 70.65 | 66.65 | 64.65 |
| Ethanol 200 pf. | 6 | 6 | 6 |
| Dimethyl Isosorbide | 1 | 1 | 1 |
| PPG-2 Hydroxyethyl Cocamide | 5 | 5 | 5 |
| Ethyl 4-(dimethyl amino) | 0.3 | 0.3 | 0.3 |
| benzoate | | | |
| Silica/13.3% BPO | 6.6 | 6.6 | 6.6 |

Formulations 11-14. Exemplary formulations which demonstrate control time to onset of polymerization: See, e.g., FIG. 1.

| | (11) | (12) | (13) | (14) |
|---|---|---|---|---|
| | % by | % by | % by | % by |
| Ingredient | weight | weight | weight | weight |
| Dimethyl Amino Methyl Propanol | 0.92 | 0.68 | 0.95 | 0 |
| Tetraethylene glycol | 8 | 8 | 8 | 8 |
| dimethacrylate | | | | |
| 2-(Acetoacetoxy)ethyl | 3 | 4 | 4 | 4 |
| methacrylate) | | | | |
| Cetyl Methacrylate | 69.59 | 69.1 | 69.03 | 69.72 |
| Ethanol 200 pf. | 6 | 6 | 6 | 6 |
| Dimethyl Isosorbide | 1 | 1 | 1 | 1 |
| Cocamide MEA | 1 | 0 | 0 | 2 |
| PPG-2 Hydroxyethyl Cocamide | 3 | 4 | 3.7 | 2 |
| Lauric acid | 0 | 0 | 0.05 | 0 |
| Ethyl 4-(dimethyl amino) | 0.69 | 0.62 | 0.67 | 0.68 |
| benzoate | | | | |
| Silica/13.3% BPO | 6.8 | 6.6 | 6.6 | 6.6 |

Formulations 15-17. Exemplary formulations for increasing concentration of surfactant improves surface wetting/penetration. See, e.g., FIG. 2.

| | 15 | 16 | 17 |
|---|---|---|---|
| | (% by | (% by | (% by |
| Ingredient | weight) | weight) | weight) |
| Dimethyl Amino Methyl Propanol | 1.07 | 1.07 | 1.07 |
| Tetraethylene glycol dimethacrylate | 8 | 8 | 8 |
| Stearyl Methacrylate | 70.03 | 70.23 | 69.23 |
| Ethanol 200 pf. | 6 | 6 | 6 |
| Dimethyl Isosorbide | 1 | 1 | 1 |
| Cocamide MEA | 0.2 | 0 | 1 |
| Ethyl 4-(dimethyl amino) benzoate | 0.7 | 0.7 | 0.7 |
| Silica/9.4% BPO | 13 | 13 | 13 |

Formulations 18-20. Exemplary formulations demonstrating the effects of formulations with increasing concentrations of the cross-linker triethylene glycol dimethacrylate (TEGDMA).

| | 18 | 19 | 20 |
|---|---|---|---|
| | (% by | (% by | (% by |
| Ingredient | weight) | weight) | weight) |
| Dimethyl Amino Methyl Propanol | 1.08 | 1.08 | 0.92 |
| Tetraethylene glycol dimethacrylate | 0 | 8 | 16 |
| Stearyl Methacrylate | 78.02 | 70.02 | 62.43 |
| Ethanol 200 pf. | 6 | 6 | 6 |
| Dimethyl Isosorbide | 1 | 1 | 1 |
| Cocamide MEA | 0.2 | 0.2 | 0.2 |
| Ethyl 4-(dimethyl amino) benzoate | 0.7 | 0.7 | 0.45 |
| Silica/9.4% BPO | 13 | 13 | 13 |

Formulation 21. Exemplary formulation demonstrating the rate of absorption of this low alkalinity formulation into the hair over time. See, e.g., FIG. 4.

| | (21) |
|---|---|
| Ingredient | % by weight |
| Dimethyl Amino Methyl Propanol | 0.2 |
| Tetraethylene glycol dimethacrylate | 8.0 |
| 2-(Acetoacetoxy)ethyl methacrylate) | 5.0 |
| Cetyl Methacrylate | 72.93 |
| Ethanol 200 pf. | 4.5 |
| Dimethyl Isosorbide | 1.2 |
| POE (20) Sorbitan Monolaurate | 2.0 |
| PPG-2 Hydroxyethyl Cocamide | 4.7 |
| Ethyl 4-(dimethyl amino) benzoate | 0.47 |
| Phytantriol | 1 |
| Silica/15% BPO | 8.0 |

Formulation 22. Exemplary formulation demonstrating the use of a trifunctional cross-linker.

| | (22) |
|---|---|
| Ingredient | % by weight |
| Dimethyl Amino Methyl Propanol | 0.41 |
| Trimethylolpropane trimethacrylate | 4.59 |
| 2-(Acetoacetoxy)ethyl methacrylate) | 4.59 |
| Cetyl Methacrylate | 70.92 |
| Ethanol 200 pf. | 5.5 |
| Dimethyl Isosorbide | 0.92 |
| PPG-2 Hydroxyethyl Cocamide | 4.6 |
| Ethyl 4-(dimethyl amino) benzoate | 0.275 |
| Silica/15% BPO powder | 8.2 |

Formulation 23. Exemplary formulation demonstrating water as the carrier for BPO.

| | (23) |
|---|---|
| Ingredient | % by weight |
| Dimethyl Amino Methyl Propanol | 0.29 |
| Trimethylolpropane trimethacrylate | 7.8 |
| 2-(Acetoacetoxy)ethyl methacrylate) | 4.9 |
| Cetyl Methacrylate | 70.91 |
| Ethanol 200 pf. | 5.862 |
| Dimethyl Isosorbide | 0.98 |
| PPG-2 Hydroxyethyl Cocamide | 6.5 |
| Ethyl 4-(dimethyl amino) benzoate | 0.47 |
| 53.9% BPO/Water | 2.3 |

Formulation 24. Exemplary formulation demonstrating a silica blend designed to have significantly lower dusts formulation when used as the carrier for BPO.

| | | | (24) |
|---|---|---|---|
| | Ingredient | | % by weight |
| Dimethyl Amino Methyl Propanol | | | 0.26 |
| Trimethylolpropane trimethacrylate | | | 6.9 |
| 2-(Acetoacetoxy)ethyl methacrylate) | | | 4.3 |
| Cetyl Methacrylate | | | 62.9 |
| Ethanol 200 pf. | | | 5.2 |
| Dimethyl Isosorbide | | | 0.86 |
| PPG-2 Hydroxyethyl Cocamide | | | 5.8 |
| Ethyl 4-(dimethyl amino) benzoate | | | 0.32 |
| BPO/Water/Silica powder blend | | | 13.5 |
| | Benzoyl peroxide | 6.7% | |
| | Silica | 71.3% | |
| | Water | 22% | |

Formulation 25. Exemplary basic formulation with no crosslinker or alkalizer.

| | (25) |
|---|---|
| Ingredient | % by weight |
| Cetyl/Stearyl Methacrylate | 46 |
| Ethanol 200 pf. | 3.05 |
| Dimethyl 9-Decenamide | 0.95 |
| Acrylic powder/2.7% BPO | 50 |

Formulation 26. Exemplary formulation using 75% BPO in water as the initiator and no alkalizing agent with an induction period of 1.8 hours.

| | (26) |
|---|---|
| Ingredient | % by weight |
| Tetraethylene glycol dimethacrylate | 8.0 |
| 2-(Acetoacetoxy)ethyl methacrylate) | 5.0 |
| Cetyl Methacrylate | 71.1 |
| Ethanol 200 pf. | 6.0 |
| Dimethyl Isosorbide | 1.0 |
| PPG-2 Hydroxyethyl Cocamide | 6.8 |
| Ethyl 4-(dimethyl amino) benzoate | 0.5 |
| 75% BPO/Water | 1.6 |

Formulation 27. Exemplary formulation demonstrating curing without a catalyst

| | (26) |
|---|---|
| Ingredient | % by weight |
| Dimethyl Amino Methyl Propanol | 0.45 |
| Tetraethylene glycol dimethacrylate | 8 |
| 2-(Acetoacetoxy)ethyl methacrylate) | 4 |
| Cetyl Methacrylate | 68.1 |
| Ethanol 200 pf. | 6 |
| Dimethyl Isosorbide | 1 |
| PPG-2 Hydroxyethyl Cocamide | 4.25 |
| Silica/13% BPO powder | 8.2 |

Formulation 28. Exemplary formulation demonstrating the use of an alternative surfactant.

| | (26) |
|---|---|
| Ingredient | % by weight |
| Dimethyl Amino Methyl Propanol | 0.2 |
| Tetraethylene glycol dimethacrylate | 8 |
| 2-(Acetoacetoxy)ethyl methacrylate) | 5 |
| Cetyl Methacrylate | 64.6 |
| Ethanol 200 pf. | 6 |
| Dimethyl Isosorbide | 1 |
| POE (20) Sorbitan Monolaurate | 6.7 |
| Ethyl 4-(dimethyl amino) benzoate | 0.28 |
| Silica/15% BPO powder | 8.2 |

Formulation 29. Exemplary UV photopolymerizable formulation.

| Ingredient | % by weight |
|---|---|
| Dimethyl Amino Methyl Propanol | 0.20 |
| Tetraethylene glycol dimethacrylate | 7.90 |
| 2-(Acetoacetoxy)ethyl methacrylate | 4.93 |
| Cetyl Methacrylate | 71.83 |
| Ethanol 200 pf. | 4.43 |
| Dimethyl Isosorbide | 1.18 |
| POE (20) Sorbitan Monolaurate | 1.97 |
| PPG-2 Hydroxyethyl Cocamide | 4.63 |
| Ethyl 4-(dimethyl amino) benzoate | 0.46 |
| Phytantriol | 0.99 |
| TPO-L | 1.48 |

Formulation 30. Exemplary UV photopolymerizable formulation without a volatile organic solvent.

| Ingredient | % by weight |
|---|---|
| Dimethyl Amino Methyl Propanol | 0.20 |
| Tetraethylene glycol dimethacrylate | 8.0 |
| 2-(Acetoacetoxy)ethyl methacrylate | 5.0 |
| Cetyl Methacrylate | 76.33 |
| Dimethyl Isosorbide | 1.2 |
| POE (20) Sorbitan Monolaurate | 1.8 |
| PPG-2 Hydroxyethyl Cocamide | 4.7 |
| Ethyl 4-(dimethyl amino) benzoate | 0.47 |
| Phytantriol | 1.0 |
| TPO-L | 1.5 |

Formulation 31. Exemplary formulation demonstrating curing without a catalyst or synergist.

| Ingredient | % by weight |
|---|---|
| Dimethyl Amino Methyl Propanol | 0.20 |
| Tetraethylene glycol dimethacrylate | 7.90 |
| 2-(Acetoacetoxy)ethyl methacrylate | 4.93 |
| Cetyl Methacrylate | 71.77 |
| Ethanol 200 pf. | 4.43 |
| Dimethyl Isosorbide | 1.18 |
| POE (20) Sorbitan Monolaurate | 1.97 |
| PPG-2 Hydroxyethyl Cocamide | 4.63 |
| Phytantriol | 0.99 |
| TPO-L | 2.0 |

Formulation 32. Exemplary dual UV/Visible polychromatic photopolymerizable formulation.

| Ingredient | % by weight |
|---|---|
| Dimethyl Amino Methyl Propanol | 0.20 |
| Tetraethylene glycol dimethacrylate | 7.90 |
| 2-(Acetoacetoxy)ethyl methacrylate | 4.93 |
| Cetyl Methacrylate | 69.76 |
| Ethanol 200 pf. | 4.43 |
| Dimethyl Isosorbide | 1.18 |
| POE (20) Sorbitan Monolaurate | 1.97 |
| PPG-2 Hydroxyethyl Cocamide | 4.63 |
| Ethyl 4-(dimethyl amino) benzoate | 0.46 |
| Phytantriol | 0.99 |
| TPO-L | 2.5 |
| Camphorquinone | 1.25 |

Formulation 33. Exemplary visible light cure photopolymerizable formulation

| Ingredient | % by weight |
|---|---|
| Dimethyl Amino Methyl Propanol | 0.20 |
| Tetraethylene glycol dimethacrylate | 7.90 |
| 2-(Acetoacetoxy)ethyl methacrylate | 4.93 |
| Cetyl Methacrylate | 71.83 |
| Ethanol 200 pf. | 4.43 |
| Dimethyl Isosorbide | 1.18 |
| POE (20) Sorbitan Monolaurate | 1.97 |
| PPG-2 Hydroxyethyl Cocamide | 4.63 |
| Ethyl 4-(dimethyl amino) benzoate | 0.46 |
| Phytantriol | 0.99 |
| Camphorquinone | 2.5 |

Formulations 34 A, B and C. Exemplary formulations using different concentrations of photoinitiator in a photopolymerizable composition

| | % by | % by | % by |
|---|---|---|---|
| | weight | weight | weight |
| Ingredient | Formula A | Formula B | Formula C |
| Dimethyl Amino Methyl Propanol | 0.20 | 0.20 | 0.20 |
| Tetraethylene glycol dimethacrylate | 8.0 | 8.0 | 8.0 |
| 2-(Acetoacetoxy)ethyl methacrylate | 5.0 | 5.0 | 5.0 |
| Cetyl Methacrylate | 78.13 | 76.13 | 75.63 |
| Dimethyl Isosorbide | 1.2 | 1.2 | 1.2 |
| PPG-2 Hydroxyethyl Cocamide | 6.0 | 6.0 | 6.0 |
| Ethyl 4-(dimethyl amino) benzoate | 0.47 | 0.47 | 0.47 |
| Phytantriol | 1.0 | 1.0 | 1.0 |
| TPO-L | 0 | 2.0 | 2.5 |

Formulations 35 A and B. Exemplary formulations comparing dual thermal/photoinitiator curing to UV curing applied as treatments before and after bleaching hair.

| | % by weight | % by weight |
|---|---|---|
| Ingredient | Formula A | Formula B |
| Dimethyl Amino Methyl Propanol | 0.20 | 0.20 |
| Tetraethylene glycol dimethacrylate | 8.0 | 8.0 |
| 2-(Acetoacetoxy)ethyl methacrylate | 5.0 | 5.0 |
| Cetyl Methacrylate | 78.13 | 76.13 |
| Dimethyl Isosorbide | 1.2 | 1.2 |
| PPG-2 Hydroxyethyl Cocamide | 6.0 | 6.0 |
| Ethyl 4-(dimethyl amino) benzoate | 0.47 | 0.47 |
| Phytantriol | 1.0 | 1.0 |
| TPO-L | 1.5 | 1.5 |
| Silica/15% BPO powder | 0 | 1.5 |

1. A hair treatment composition comprising:
at least one (meth)acrylate monomer;
optionally at least one di(meth)acrylate monomer;
optionally at least one tri(meth)acrylate monomer;
a polymerization initiator;
optionally a polymerization catalyst and/or synergist;
optionally a surfactant;
optionally a pH adjuster;
optionally a penetration enhancer;
optionally a carrier;
optionally a colorant; and
optionally a conditioning agent,
wherein the (meth)acrylate monomer, the di(meth)acrylate and/or the tri(meth)acrylate monomers are present in the composition in a total amount of least 40% by weight of the total composition, and
wherein hair treated by the composition shows an improvement of at least 5% in energy to break.

2. The hair treatment composition according to claim 1, wherein the composition comprises one (meth)acrylate monomer and one di(meth)acrylate monomer.

3.-4. (canceled)

5. The hair treatment composition according to claim 1, wherein the polymerization initiator is at least one selected from a UV light sensitive photoinitiator, a visible light sensitive photoinitiator, and a polychromatic light sensitive photoinitiator.

6. The hair treatment composition according to claim 1, wherein the polymerization initiator is a thermal initiator.

7. The hair treatment composition according to claim 1, wherein the surfactant is present and is an alkoxylated amide and/or an ethoxylated fatty acid.

8.-9. (canceled)

10. The hair treatment composition according to claim 1, wherein at least two of the di(meth)acrylate monomer, the tri(meth)acrylate monomer, the surfactant, the penetration enhancer and the carrier are present.

11. The hair treatment composition according to claim 1, wherein at least three of the di(meth)acrylate monomer, the tri(meth)acrylate monomer, the surfactant, the penetration enhancer and the carrier are present.

12. The hair treatment composition according to claim 1, wherein the di(meth)acrylate monomer and at least one of the surfactant, the penetration enhancer and the carrier is present.

13. The hair treatment composition according to claim 1, wherein the di(meth)acrylate monomer and at least two of the surfactant, the penetration enhancer and the carrier are present.

14. The hair treatment composition according to claim 1, wherein the di(meth)acrylate monomer, the surfactant, the penetration enhancer and the carrier are present.

15. (canceled)

16. The hair treatment composition according to claim 1, wherein hair treated by the composition also shows an improvement of at least 5% in elastic modulus.

17. (canceled)

18. A hair treatment composition comprising:
10 to 99% by weight of at least one (meth)acrylate monomer;
5 to 20% by weight of at least one di(meth)acrylate monomer; and/or
1 to 5% by weight of at least one tri(meth)acrylate monomer;
between 0 and up to 5% by weight of a photoinitiator;
0 to 2% by weight of a polymerization catalyst;
0 to 15% by weight of a surfactant;
0 to 20% by weight of a penetration enhancer;
0 to 5% by weight of an alkalizing agent and/or pH adjuster;
0 to 40% by weight of an organic solvent;
0 to 50% by weight of a carrier;
0 to 5% by weight of a colorant and/or optical brightener; and
0 to 5% by weight of a conditioning agent,
wherein the (meth)acrylate monomer and the optional di(meth)acrylate and tri(meth)acrylate monomers are present in the composition in a total amount of at least 40% by weight of the total composition, and
wherein hair treated by the composition shows an improvement of at least 5% in energy to break.

19. A method for treating hair, comprising applying the hair treatment composition of claim 1 to hair.

20.-22. (canceled)

23. The method according to claim 19, wherein at least a second treatment of the hair with the composition results in cumulative improvement in the energy to break.

24. The method according to claim 19, wherein at least a second treatment of the hair with the composition results in cumulative improvement in the elastic modulus.

25.-29. (canceled)

30. A kit comprising the hair treatment composition of claim 1 and instructions for use.

31. The method of claim 19, wherein the hair treatment composition is applied before, after or simultaneously with another composition for improving hair health.

32. The method of claim 19, wherein the hair treatment composition is applied as a standalone service to improve hair health and not in combination with other salon hair services, such as bleaching, tinting, coloring, permanent waving or straightening or curling.

33. The hair treatment composition according to claim 1, selected from:
dimethyl amino methyl propanol, tetraethylene glycol dimethacrylate, 2-(acetoacetoxy)ethyl methacrylate, cetyl methacrylate, ethanol, dimethyl isosorbide, PPG-2-hydroxyethyl cocoamide, ethyl 4-(dimethyl amino)benzoate, benzoyl peroxide, and silica;
dimethyl amino methyl propanol, 1,12-dodecanediol dimethacrylate, stearyl methacrylate, ethanol, dimethyl isosorbide, cocoamide MEA, ethyl 4-(dimethyl amino) benzoate, benzoyl peroxide, and silica;
dimethyl amino methyl propanol, 2-(acetoacetoxy)ethyl methacrylate, cetyl methacrylate, ethanol, dimethyl isosorbide, PPG-2 hydroxyethyl cocoamide ethyl 4-(dimethyl amino)benzoate, benzoyl peroxide, and silica;
dimethyl amino methyl propanol, 1,12-dodecanediol dimethacrylate, 2-(acetoacetoxy)ethyl methacrylate, cetyl methacrylate, ethanol, dimethyl isosorbide, PPG-2 hydroxyethyl coco/isostearamide, ethyl 4-(dimethyl amino)benzoate, benzoyl peroxide, and silica;
dimethyl amino methyl propanol, tetraethylene glycol dimethacrylate, 2-(acetoacetoxy)ethyl methacrylate, cetyl methacrylate, ethanol, dimethyl isosorbide, PPG-2-hydroxyethyl cocoamide, ethyl 4-(dimethyl amino)benzoate, benzoyl peroxide, and silica;
tetraethylene glycol dimethacrylate, stearyl methacrylate, ethanol, dimethyl isosorbide, PPG-2-hydroxyethyl cocoamide, ethyl 4-(dimethyl amino)benzoate, benzoyl peroxide, and silica;
dimethyl amino methyl propanol, cetyl methacrylate, ethanol, dimethyl isosorbide, cocoamide MEA, ethyl 4-(dimethyl amino)benzoate, benzoyl peroxide, and silica;
cetyl/stearyl methacrylate, ethanol, dimethyl 9-decenamide, benzoyl peroxide, and silica;
dimethyl amino methyl propanol, tetraethylene glycol dimethacrylate, 2-(acetoacetoxy)ethyl methacrylate, cetyl methacrylate, ethanol, dimethyl isosorbide, cocoamide MEA, PPG-2-hydroxyethyl cocoamide, ethyl 4-(dimethyl amino)benzoate, benzoyl peroxide, and silica;
dimethyl amino methyl propanol, tetraethylene glycol dimethacrylate, 2-(acetoacetoxy)ethyl methacrylate, cetyl methacrylate, ethanol, dimethyl isosorbide, cocoamide MEA, PPG-2-hydroxyethyl cocoamide, lauric acid, ethyl 4-(dimethyl amino) benzoate, benzoyl peroxide, and silica;
dimethyl amino methyl propanol, tetraethylene glycol dimethacrylate, stearyl methacrylate, ethanol, dimethyl isosorbide, cocoamide MEA, ethyl 4-(dimethyl amino) benzoate, benzoyl peroxide, and silica;
dimethyl amino methyl propanol, tetraethylene glycol dimethacrylate, 2-(acetoacetoxy)ethyl methacrylate, cetyl methacrylate, ethanol, dimethyl isosorbide, PPG-2-hydroxyethyl cocoamide, ethyl 4-(dimethyl amino)benzoate, benzoyl peroxide, and silica;
dimethyl amino methyl propanol, trimethylolpropane trimethacrylate, 2-(acetoacetoxy)ethyl methacrylate, cetyl methacrylate, ethanol, dimethyl isosorbide, PPG-2 hydroxyethyl cocoamide, ethyl 4-(dimethyl amino)benzoate, benzoyl peroxide, and silica;
cetyl/stearyl methacrylate, ethanol, dimethyl 9-decenamide, benzoyl peroxide and acrylic powder;
tetraethylene glycol dimethacrylate, 2-(acetoacetoxy)ethyl methacrylate, cetyl methacrylate, ethanol, dimethyl isosorbide, PPG-2 hydroxyethyl cocoamide, ethyl 4-(dimethyl amino)benzoate, benzoyl peroxide and water;
dimethyl amino methyl propanol, tetraethylene glycol dimethacrylate, 2-(acetoacetoxy)ethyl methacrylate, cetyl methacrylate, ethanol, dimethyl isosorbide, PPG-2 hydroxyethyl cocoamide, benzoyl peroxide, and silica;
dimethyl amino methyl propanol, tetraethylene glycol dimethacrylate, 2-(acetoacetoxy)ethyl methacrylate, cetyl methacrylate, ethanol, dimethyl isosorbide, POE (20) sorbitan monolaurate, ethyl 4-(dimethyl amino)benzoate, benzoyl peroxide, and silica;
dimethyl amino methyl propanol, tetraethylene glycol dimethacrylate, 2-(acetoacetoxy)ethyl methacrylate, cetyl methacrylate, ethanol, dimethyl isosorbide, POE (20) sorbitan monolaurate, PPG-2 hydroxyethyl cocamide, ethyl 4-(dimethyl amino) benzoate, phytantriol, and TPO-L;
dimethyl amino methyl propanol, tetraethylene glycol dimethacrylate, 2-(acetoacetoxy)ethyl methacrylate, cetyl methacrylate, dimethyl isosorbide, POE (20) sorbitan monolaurate, PPG-2 hydroxyethyl cocamide, ethyl 4-(dimethyl amino) benzoate, phytantriol, and TPO-L;
dimethyl amino methyl propanol, tetraethylene glycol dimethacrylate, 2-(acetoacetoxy)ethyl methacrylate, cetyl methacrylate, ethanol, dimethyl isosorbide, POE (20) sorbitan monolaurate, PPG-2 hydroxyethyl cocamide, ethyl 4-(dimethyl amino) benzoate, phytantriol, TPO-L, and camphorquinone;
dimethyl amino methyl propanol, tetraethylene glycol dimethacrylate, 2-(acetoacetoxy)ethyl methacrylate, cetyl methacrylate, ethanol, dimethyl isosorbide, POE (20) sorbitan monolaurate, PPG-2 hydroxyethyl cocamide, ethyl 4-(dimethyl amino) benzoate, phytantriol, and camphorquinone;
dimethyl amino methyl propanol, tetraethylene glycol dimethacrylate, 2-(acetoacetoxy)ethyl methacrylate, cetyl methacrylate, dimethyl isosorbide, PPG-2 hydroxyethyl cocamide, ethyl 4-(dimethyl amino)benzoate, phytantriol, and TPO-L; and
dimethyl amino methyl propanol, tetraethylene glycol dimethacrylate, 2-(acetoacetoxy)ethyl methacrylate, cetyl methacrylate, dimethyl isosorbide, PPG-2 hydroxyethyl cocamide, ethyl 4-(dimethyl amino)benzoate, phytantriol, TPO-L, benzoyl peroxide, and silica.

34. A method of improving hair health, comprising of applying the hair treatment composition of claim 1 to hair allowing sufficient time for the hair to absorb a portion of the applied composition into the shafts of the hair, where the monomer components of the composition polymerize inside the shafts to improve hair health; and rinsing the hair to remove residual applied composition present on the hair exterior.
